SEQUENCE LISTING (87 sequences)

SEQ ID NO: 1 - protein, 851 aa, synthetic construct
MEAMLPLFEPKGRVLLVDGHHLAYRTFFALKGLTTSRGEPVQAVYGFAKSLLKALKEDGYKAVFVVFDAKAPSFRHEAYEAYKAGRAPTPEDFPRQLALIKELVDLLGFTRLEVPGYEADDVLATLAKKAEKEGYEVRILTADRDLYQLVSDRVAVLHPEGHLITPEWLWEKYGLRPEQWVDFRALVGDPSDNLPGVKGIGEKTALKLLKEWGSLENLLKNLDRVKPENVREKIKAHLEDLRLSLELSRVRTDLPLEVDLAQGREPDREGLRAFLERLEFGSLLHEFGLLEAPAPLEEAPWPPPEGAFVGFVLSRPEPMWAELKALAACRDGRVHRAADPLAGLKDLKEVRGLLAKDLAVLASREGLDLVPGDDPMLLAYLLDPSNTTPEGVARRYGGEWTEDAAHRALLSERLHRNLLKRLEGEEKLLWLYHEVEKPLSRVLAHMEATGVRLDVAYLQALSLELAEEIRRLEEEVFRLAGHPFNLNSRDQLERVLFDELRLPALKKTKKTGKRSTSAAVLEALREAHPIVEKILQHRELTKLKNTYVDPLPSLVHPRTGRLHTRFNQTATATGRLSSSDPNLQNIPVRTPLGQRIRRAFVAEVGWALVVLDYSQMGLRVLAHLSGDENLIRVFQEGKDIHTQTASWMFGVPPEAVDPLMRRAAKTVNFGVLYGMSAHRLSQELAIPYEEAVAFIERYFQSFPKVRAWIEKTLEEGRKRGYVETLFGRRRYVPDLNARVKSVREAAERMAFNMPVQGTAADLMKLAMVKLFPRLREMGARMLLQVADELLLEAPQARAEEVAALAKEAMEKAYPLAVPLEVEVGMGEDWLSAKGVSGWRLFKKISHHHHHH

SEQ ID NO: 2 - protein, 851 aa, synthetic construct
MEAMLPLFEPKGRVLLVDGHHLAYRTFFALKGLTTSRGEPVQAVYGFAKSLLKALKEDGYKAVFVVFDAKAPSFRHEAYEAYKAGRAPTPEDFPRQLALIKELVDLLGFTRLEVPGYEADDVLATLAKKAEKEGYEVRILTADRDLYQLVSDRVAVLHPEGHLITPEWLWEKYGLRPEQWVDFRALVGDPSDNLPGVKGIGEKTALKLLKEWGSLENLLKNLDRVKPENVREKIKAHLEDLRLSLELSRVRTDLPLEVDLAQGREPDREGLRAFLERLEFGSLLHEFGLLEAPAPLEEAPWPPPEGAFVGFVLSRPEPMWAELKALAACRDGRVHRAADPLAGLKDLKEVRGLLAKDLAVLASREGLDLVPGDDPMLLAYLLDPSNTTPEGVARRYGGEWTEDAAHRALLSERLHRNLLKRLEGEEKLLWLYHEVEKPLSRVLAHMEATGVRLDVAYLQALSLELAEEIRRLEEEVFRLAGHPFNLNSRDQLERVLFDELRLPALKKTKKTGKRSTSAAVLEALREAHPIVEKILQHRELTKLKNTYVDPLPSLVHPRTGRLHTRFNQTATATGRLSSSDPNLQNIPVRTPLGQRIRRTFVAEAGRQLVALDYSQTELRVLAHLSGDENLIRVFQEGKDIHTQTASWMFGVPPEAVDPLMRRAAKTVNFGVLYGMSAHRLSQELAIPYEEAVAFIERYFQSFPKVRAWIEKTLEEGRKRGYVETLFGRRRYVPDLNARVKSVREAAERMAFNMPVQGTAADLMKLAMVKLFPRLREMGARMLLQVADELLLEAPQARAEEVAALAKEAMEKAYPLAVPLEVEVGMGEDWLSAKGVSGWRLFKKISHHHHHH

SEQ ID NO: 3 - DNA, 45 bp, synthetic construct
  source 1..45: mol_type = other DNA
taatttctactaagtgtagatcaacgtcgtgactgggaaaaccct

SEQ ID NO: 4 - DNA, 30 bp, synthetic construct
  source 1..30: mol_type = other DNA
cacgacgttgtaaaacgacggccagtgaat

SEQ ID NO: 5 - DNA, 40 bp, synthetic construct
  source 1..40: mol_type = other DNA
gccgtcgttttacaacgtcgtgnctgggaaaaccctggcg

SEQ ID NO: 6 - DNA, 45 bp, synthetic construct
  source 1..45: mol_type = other DNA
cgccagggttttcccagncacgacgttgtaaaacgacggccagtg

SEQ ID NO: 7 - DNA, 18 bp, synthetic construct
  source 1..18: mol_type = other DNA
cgccagggttttcccagt

SEQ ID NO: 8 - DNA, 10 bp, synthetic construct
  source 1..10: mol_type = other DNA
ttattattat

SEQ ID NO: 9 - DNA, 43 bp, synthetic construct
  source 1..43: mol_type = other DNA
aaatttaccaagcaggcagtaagccatgtaacggagtagaggg

SEQ ID NO: 10 - DNA, 47 bp, synthetic construct
  source 1..47: mol_type = other DNA
ttactgcctgcttggtaaatttcggttgaaatatcccgctcaaaagg

SEQ ID NO: 11 - DNA, 23 bp, synthetic construct
  source 1..23: mol_type = other DNA
taatacgactcactatagggaga

SEQ ID NO: 12 - DNA, 18 bp, synthetic construct
  source 1..18: mol_type = other DNA
gatcagcgggtttaaacg

SEQ ID NO: 13 - DNA, 90 bp, synthetic construct
  source 1..90: mol_type = other DNA
atgaagttgctgatggtcctcatgctggcggccctctcccagcactgctacgcaggctctggctgccccttattggagaatgtgatttcc

SEQ ID NO: 14 - DNA, 90 bp, synthetic construct
  source 1..90: mol_type = other DNA
tgtcgtctatgaactcttgaagaagttctttgtattcagtcttagacacttgtggattgattgtcttggaaatcacattctccaataagg

SEQ ID NO: 15 - DNA, 90 bp, synthetic construct
  source 1..90: mol_type = other DNA
tcaagagttcatagacgacaatgccactacaaatgccatagatgaattgaaggaatgttttcttaaccaaacggatgaaactctgagcaa

SEQ ID NO: 16 - DNA, 74 bp, synthetic construct
  source 1..74: mol_type = other DNA
ttaaaataaatcacaaagactgctgtcatatattaattgcataaacacctcaacattgctcagagtttcatccg

SEQ ID NO: 17 - DNA, 38 bp, synthetic construct
  source 1..38: mol_type = other DNA
taatacgactcactatagggatgaagttgctgatggtc

SEQ ID NO: 18 - DNA, 27 bp, synthetic construct
  source 1..27: mol_type = other DNA
ttaaaataaatcacaaagactgctgtc

SEQ ID NO: 19 - DNA, 15 bp, synthetic construct
  source 1..15: mol_type = other DNA
ctgattgtcctcact

SEQ ID NO: 20 - DNA, 45 bp, synthetic construct
  source 1..45: mol_type = other DNA
atctctaattgaggttgaacctcaacaattgtttgaatagtagta

SEQ ID NO: 21 - DNA, 59 bp, synthetic construct
  source 1..59: mol_type = other DNA
catctctaattgaggttgaacctcaacaattgtttgaatagtagaccgtcacgccagtc

SEQ ID NO: 22 - DNA, 44 bp, synthetic construct
  source 1..44: mol_type = other DNA
gttgtctgattgtcctcactgcgaagagcaagaagaagattggt

SEQ ID NO: 23 - DNA, 42 bp, synthetic construct
  source 1..42: mol_type = other DNA
atgcagacattgtggaagaagctgcaacacctcctccatgtt

SEQ ID NO: 24 - DNA, 21 bp, synthetic construct
  source 1..21: mol_type = other DNA
gccacttctgctgctcttcaa

SEQ ID NO: 25 - DNA, 22 bp, synthetic construct
  source 1..22: mol_type = other DNA
cttgcatggcattgttagtagc

SEQ ID NO: 26 - DNA, 22 bp, synthetic construct
  source 1..22: mol_type = other DNA
cgtcttgttgaccaacagtttg

SEQ ID NO: 27 - DNA, 22 bp, synthetic construct
  source 1..22: mol_type = other DNA
acagtggttgttaatgcagcca

SEQ ID NO: 28 - DNA, 22 bp, synthetic construct
  source 1..22: mol_type = other DNA
gttgtctgattgtcctcactgc

SEQ ID NO: 29 - DNA, 21 bp, synthetic construct
  source 1..21: mol_type = other DNA
tgcagacattgtggaagaagc

SEQ ID NO: 30 - DNA, 43 bp, synthetic construct
  source 1..43: mol_type = other DNA
accaacagtttgttgactatcatcactgctgctcttcaacctg

SEQ ID NO: 31 - DNA, 41 bp, synthetic construct
  source 1..41: mol_type = other DNA
cggcagtgaggacaatcagacactggtgtaagttccatctc

SEQ ID NO: 32 - DNA, 18 bp, synthetic construct
  source 1..18: mol_type = other DNA
tggaatttggtgccactt

SEQ ID NO: 33 - DNA, 24 bp, synthetic construct
  source 1..24: mol_type = other DNA
ctattcacttcaatagtctgaaca

SEQ ID NO: 34 - DNA, 22 bp, synthetic construct
  source 1..22: mol_type = other DNA
caatcttcttcttgctcttctt

SEQ ID NO: 35 - DNA, 25 bp, synthetic construct
  source 1..25: mol_type = other DNA
ttcaaacaattgttgaggttcaacc

SEQ ID NO: 36 - DNA, 45 bp, synthetic construct
  source 1..45: mol_type = other DNA
caatagtctgaacaactggtgtaagtcaaacaattgttgaggttc

SEQ ID NO: 37 - DNA, 40 bp, synthetic construct
  source 1..40: mol_type = other DNA
tgcagacattgtggaagaagcattggctgcattaacaacc

SEQ ID NO: 38 - DNA, 19 bp, synthetic construct
  source 1..19: mol_type = other DNA
tgaggacaatcagacaact

SEQ ID NO: 39 - DNA, 20 bp, synthetic construct
  source 1..20: mol_type = other DNA
ctcctccatgtttaaggtaa

SEQ ID NO: 40 - DNA, 18 bp, synthetic construct
  source 1..18: mol_type = other DNA
ccatctctaattgaggtt

SEQ ID NO: 41 - DNA, 17 bp, synthetic construct
  source 1..17: mol_type = other DNA
aaggtaaaaccaacagt

SEQ ID NO: 42 - DNA, 46 bp, synthetic construct
  source 1..46: mol_type = other DNA
ccttggtaatcatcttcagtaccatgaagaagaaggtgattgtgaa

SEQ ID NO: 43 - DNA, 46 bp, synthetic construct
  source 1..46: mol_type = other DNA
ttggaatttggtgccacttctgtttgttgactatcatcatctaacc

SEQ ID NO: 44 - DNA, 19 bp, synthetic construct
  source 1..19: mol_type = other DNA
ctaccctccagatgaggat

SEQ ID NO: 45 - DNA, 18 bp, synthetic construct
  source 1..18: mol_type = other DNA
cactgccgtcttgttgac

SEQ ID NO: 46 - DNA, 25 bp, synthetic construct
  source 1..25: mol_type = other DNA
ctcatattgagttgatggctcaaac

SEQ ID NO: 47 - DNA, 23 bp, synthetic construct
  source 1..23: mol_type = other DNA
caacctgaagaagagcaagaaga

SEQ ID NO: 48 - DNA, 28 bp, synthetic construct
  source 1..28: mol_type = other DNA
ggaacttacaccagttgttcagactatt

SEQ ID NO: 49 - DNA, 26 bp, synthetic construct
  source 1..26: mol_type = other DNA
tagcttcttccacaatgtctgcattt

SEQ ID NO: 50 - DNA, 26 bp, synthetic construct
  source 1..26: mol_type = other DNA
gctgtcgatgacaggttgttacaaag

SEQ ID NO: 51 - DNA, 29 bp, synthetic construct
  source 1..29: mol_type = other DNA
gagattgatatctttcgataccacgacct

SEQ ID NO: 52 - DNA, 24 bp, synthetic construct
  source 1..24: mol_type = other DNA
tttgagcgggatatttcaaccgaa

SEQ ID NO: 53 - DNA, 27 bp, synthetic construct
  source 1..27: mol_type = other DNA
gccataagattgaagaggaaagtagca

SEQ ID NO: 54 - DNA, 28 bp, synthetic construct
  source 1..28: mol_type = other DNA
cctctattcctttgctctcatgcttatt

SEQ ID NO: 55 - DNA, 22 bp, synthetic construct
  source 1..22: mol_type = other DNA
gagtagaccaaggcctccaagt

SEQ ID NO: 56 - DNA, 29 bp, synthetic construct
  source 1..29: mol_type = other DNA
ccttcattgacctcaactacatggtttac

SEQ ID NO: 57 - DNA, 25 bp, synthetic construct
  source 1..25: mol_type = other DNA
cattgatgacaagcttcccgttctc

SEQ ID NO: 58 - DNA, 23 bp, synthetic construct
  source 1..23: mol_type = other DNA
cccgcagccgcttgagcaaatta

SEQ ID NO: 59 - DNA, 25 bp, synthetic construct
  source 1..25: mol_type = other DNA
gccgctggccagatccatttctatc

SEQ ID NO: 60 - DNA, 24 bp, synthetic construct
  source 1..24: mol_type = other DNA
gccgggattgagcgggtaataaat

SEQ ID NO: 61 - DNA, 27 bp, synthetic construct
  source 1..27: mol_type = other DNA
cctttctcaataacaccgagcaggaag

SEQ ID NO: 62 - DNA, 28 bp, synthetic construct
  source 1..28: mol_type = other DNA
caatgccactacaaatgccatagatgaa

SEQ ID NO: 63 - DNA, 34 bp, synthetic construct
  source 1..34: mol_type = other DNA
gactgctgtcatatattaattgcataaacacctc

SEQ ID NO: 64 - DNA, 45 bp, synthetic construct
  source 1..45: mol_type = other DNA
cgccagggttttcccagaccactaaaactattcacttcaatagtc

SEQ ID NO: 65 - DNA, 43 bp, synthetic construct
  source 1..43: mol_type = other DNA
gtctgcatttttaatgtatacattgtcagtaagttttaaatac

SEQ ID NO: 66 - DNA, 33 bp, synthetic construct
  source 1..33: mol_type = other DNA
cgccagggttttcccaggccatgcccttctccc

SEQ ID NO: 67 - DNA, 34 bp, synthetic construct
  source 1..34: mol_type = other DNA
ccaggctttcgttacattttgcagctgtacgctc

SEQ ID NO: 68 - DNA, 31 bp, synthetic construct
  source 1..31: mol_type = other DNA
cgccagggttttcccaggtactgcctgcttg

SEQ ID NO: 69 - DNA, 31 bp, synthetic construct
  source 1..31: mol_type = other DNA
cgccagggttttcccagttactgcctgcttg

SEQ ID NO: 70 - DNA, 27 bp, synthetic construct
  source 1..27: mol_type = other DNA
taaatccctctactccgttacatggcc

SEQ ID NO: 71 - DNA, 40 bp, synthetic construct
  source 1..40: mol_type = other DNA
cgccagggttttcccagctcttctgaagataaagatgatg

SEQ ID NO: 72 - DNA, 24 bp, synthetic construct
  source 1..24: mol_type = other DNA
gcctccaagtggacagagaaggta

SEQ ID NO: 73 - DNA, 30 bp, synthetic construct
  source 1..30: mol_type = other DNA
cgccagggttttcccagttgacggtgccat

SEQ ID NO: 74 - DNA, 23 bp, synthetic construct
  source 1..23: mol_type = other DNA
gacaagcttcccgttctcagcca

SEQ ID NO: 75 - DNA, 30 bp, synthetic construct
  source 1..30: mol_type = other DNA
cgccagggttttcccagtacgcggcccgac

SEQ ID NO: 76 - DNA, 26 bp, synthetic construct
  source 1..26: mol_type = other DNA
tggccagatccatttctatcatgcca

SEQ ID NO: 77 - DNA, 29 bp, synthetic construct
  source 1..29: mol_type = other DNA
cgccagggttttcccagtgctggtggtgg

SEQ ID NO: 78 - DNA, 22 bp, synthetic construct
  source 1..22: mol_type = other DNA
acaccgagcaggaagttcgtcg

SEQ ID NO: 79 - DNA, 37 bp, synthetic construct
  source 1..37: mol_type = other DNA
cgccagggttttcccagtcagagtttcatccgtttgg

SEQ ID NO: 80 - DNA, 43 bp, synthetic construct
  source 1..43: mol_type = other DNA
gactgctgtcatatattaattgcataaacacctcaacattgcg

SEQ ID NO: 81 - DNA, 81 bp, synthetic construct
  source 1..81: mol_type = other DNA
ggtgttacagacgtaaaaattacatatgtaacagtcattcaaaatttattggtgattttgataagtgaagttatcagactt

SEQ ID NO: 82 - DNA, 18 bp, synthetic construct
  source 1..18: mol_type = other DNA
cgccagggttttcccaga

SEQ ID NO: 83 - DNA, 40 bp, synthetic construct
  source 1..40: mol_type = other DNA
gcggtcccaaaagggtctgtgctgcaacattttgctgccg

SEQ ID NO: 84 - DNA, 48 bp, synthetic construct
  source 1..48: mol_type = other DNA
cgccagggttttcccagacacgacgttgtaaaacgacggccagtgaat

SEQ ID NO: 85 - DNA, 24 bp, synthetic construct
  source 1..24: mol_type = other DNA
tcccaaaagggtcagtgctgcaac

SEQ ID NO: 86 - DNA, 48 bp, synthetic construct
  source 1..48: mol_type = other DNA
cgccagggttttcccagncacgacgttgtaaaacgacggccagtgaat

SEQ ID NO: 87 - DNA, 40 bp, synthetic construct
  source 1..40: mol_type = other DNA
gcggtcccaaaagggtcngtgctgcaacattttgctgccg

(19) United States
(12) Patent Application Publication
(10) Pub. No.: US 2026/0085344 A1
(43) Pub. Date: Mar. 26, 2026

(54) METHOD OF DETECTING A POLYNUCLEOTIDE ANALYTE

(71) Applicant: National University of Singapore, Singapore (SG)

(72) Inventors: Matthew Wook Chang, Singapore (SG); Nikhil Aggarwal, Singapore (SG); In Young Hwang, Singapore (SG); Hua Ling, Singapore (SG); Jee Loon Foo, Singapore (SG)

(21) Appl. No.: 18/856,243

(22) PCT Filed: Apr. 14, 2023
(86) PCT No.: PCT/SG2023/050254

§ 371 Date: Oct. 11, 2024

(30) Foreign Application Priority Data
Apr. 14, 2022 (SG) 10202203891U

(51) Int. Cl. C12Q 1/6823; C12N 9/22; C12N 15/113; C12Q 1/25; C12Q 1/34; C12Q 1/48; C12Q 1/6844; C12Q 1/6876

(52) U.S. Cl. CPC C12Q 1/6823; C12N 9/226; C12N 15/113; C12Q 1/25; C12Q 1/34; C12Q 1/48; C12Q 1/6844; C12Q 1/6876; C12Y 207/07007; C12Y 600/00; C12N 2310/20; C12Q 2600/156

(57) ABSTRACT

The invention relates generally to the field of nucleic acid detection. In particular, the specification teaches a method of detecting a polynucleotide analyte in a sample. In one aspect, the method comprises the use of a cleaving agent with flap endonuclease activity and a type V CRISPR/Cas effector protein. In another aspect, the type V CRISPR/Cas effector protein is a Cast 2 protein.

### TECHNICAL FIELD

The invention relates, in general terms, to the field of nucleic acid detection. In particular, the specification teaches a method of detecting a polynucleotide analyte in a sample.

### BACKGROUND

In vitro amplification and detection of nucleic acids has various clinical applications, especially in the diagnosis of acute and chronic conditions, including those caused by infectious diseases. Quantitative polymerase chain reaction (qPCR) and direct sequencing are reliable methods for nucleic acid detection, but these techniques require sophisticated instruments and can be costly and time-consuming to perform. Newer generations of detection assays, including the invader assay and CRISPR-based methods, seek to combine ease of use and cost efficiency, while not compromising on assay specificity and sensitivity.

The invader assay is a method for detection and quantitative analysis of DNA or RNA. It does not amplify the target of interest but rather, generates and amplifies an unrelated signal only in the presence of the correct target sequence. The assay involves generating an invasive cleavage structure in a target-dependent manner, allowing a structure-specific enzyme to cleave the invasive cleavage structure to release a signal that may be further detected. The assay involves the use of two primers, an invading primer and a flap primer. The flap primer has a 3′ portion complementary to a target and a 5′ portion that is usually unrelated to the target sequence. The 5′ portion of the flap primer that does not hybridize to the target forms a 5′ flap. The invading primer anneals to the target 5′ of the 5′ portion of the flap primer annealed to the target, and the flap primer and the invading primer overlap, creating a bifurcated overlapping structure that is considered to resemble a structure generated during strand displacement DNA synthesis. The invading primer and flap primer often overlap by one nucleotide, although a longer overlap can also be used. The bifurcated structure is cleaved to release the 5′ flap of the 5′ primer, and the released 5′ flap then functions as a signal that can be detected. Disadvantages of the invader assay include slow speed of detection, the need for an initial denaturation step and the need for a fluorescence detection system.

CRISPR-based methods include the DETECTR and SHERLOCK assays. The DETECTR assay relies on the Cas12 nuclease to detect DNA targets, while the SHERLOCK assay relies on the Cas13 nuclease to detect RNA targets. In both methods, recognition of a nucleic acid target leads to off-target cleavage that generates a signal. A disadvantage of Cas12-based assays is that they require a PAM sequence near the target sequence for recognition by the Cas nuclease, and are thus limited in the sequences they can detect. Cas13-based assays require in vitro transcription of DNA targets to RNA for detection, making detection more complex. In both cases guide RNA activity must also be optimized for the detection of each new target.

It would be desirable to overcome or ameliorate at least one of the above-described problems, or at least to provide a useful alternative.

### SUMMARY

Disclosed herein is a method of detecting a polynucleotide analyte in a sample, the method comprising: a) contacting the sample comprising the polynucleotide analyte with: i) a first nucleic acid probe comprising a 3′ portion complementary to a first portion of the polynucleotide analyte and a 5′ portion that is not complementary to and does not hybridize to the polynucleotide analyte: ii) a second nucleic acid probe comprising a 5′ portion complementary to a second portion of said polynucleotide analyte and a 3′ portion that is not complementary to and does not hybridize to the polynucleotide analyte, wherein said first portion of the polynucleotide analyte is 5′ to and contiguous with the second portion of the polynucleotide analyte; and iii) a structure-specific nucleic acid cleaving agent: wherein hybridization of the first nucleic acid probe to the first portion of the polynucleotide analyte and hybridization of the second nucleic acid probe to the second portion of the polynucleotide analyte forms a cleavage structure: wherein formation of the cleavage structure allows cleavage of the first nucleic acid probe by the cleaving agent to release a 5′ flap comprising the 5′ portion of the first nucleic acid probe that is not complementary to and does not hybridize to the polynucleotide analyte: b) ligating the 5′ flap to a nucleic acid adaptor to form an adaptor-ligated product, wherein the nucleic acid adaptor comprises a double-stranded region and a 3′overhang extension that is complementary to the 5′ flap and wherein the 3′ overhang extension hybridizes to the 5′ flap: c) contacting the adaptor-ligated product with: i) a type V CRISPR/Cas effector protein: ii) a guide RNA comprising a region that binds to the type V CRISPR/Cas effector protein and a guide sequence that is complementary to a portion of the 5′ flap and a portion of the nucleic acid adaptor that is ligated to and adjacent to the 5′ flap; and iii) a single stranded detector DNA; and d) measuring a detectable signal produced by cleavage of the single stranded detector DNA by the type V CRISPR/Cas effector protein to detect the adaptor-ligated product, thereby detecting the polynucleotide analyte in the sample.

Disclosed herein is a method of detecting a single nucleotide polymorphism (SNP) in a polynucleotide analyte in a sample, the method comprising: a) contacting the sample comprising the polynucleotide analyte with: i) a first nucleic acid probe comprising a 3′ portion complementary to a first portion of the polynucleotide analyte and a 5′ portion that is not complementary to and does not hybridize to the polynucleotide analyte: ii) a second nucleic acid probe comprising a 5′ portion complementary to a second portion of said polynucleotide analyte and a 3′ portion that is not complementary to and does not hybridize to the polynucleotide analyte, wherein said first portion of the polynucleotide analyte is 5′ to and contiguous with the second portion of the polynucleotide analyte; and iii) a structure-specific nucleic acid cleaving agent: wherein hybridization of the first nucleic acid probe to the first portion of the polynucleotide analyte and hybridization of the second nucleic acid probe to the second portion of the polynucleotide analyte forms a cleavage structure: wherein formation of the cleavage structure allows cleavage of the first nucleic acid probe by the cleaving agent to release a 5′ flap comprising the 5′ portion of the first nucleic acid probe that is not complementary to and does not hybridize to the polynucleotide analyte: b) ligating the 5′ flap to a nucleic acid adaptor to form an adaptor ligated product, wherein the nucleic acid adaptor comprises a double-stranded region and a 3′overhang extension that is complementary to the 5′ flap and wherein the 3′ overhang extension hybridizes to the 5′ flap: c) contacting the adaptor-ligated product with: i) a type V CRISPR/Cas effector protein: ii) a guide RNA comprising a region that binds to the type V CRISPR/Cas effector protein and a guide sequence that is complementary to a portion of the 5′ flap and a portion of the nucleic acid adaptor that is ligated to and adjacent to the 5′ flap; and iii) a single stranded detector DNA; and d) measuring a detectable signal produced by cleavage of the single stranded detector DNA by the type V CRISPR/Cas effector protein to detect the adaptor-ligated product, thereby detecting the SNP in the polynucleotide analyte in the sample.

Disclosed herein is a method of detecting a polynucleotide analyte in a sample, the method comprising
a) i) contacting the sample comprising the polynucleotide analyte with a first nucleic acid probe and a second nucleic acid probe that are configured to form a cleavage structure in the presence of the polynucleotide analyte; and
   ii) a structure-specific nucleic acid cleaving agent:
wherein formation of the cleavage structure allows cleavage of the first nucleic acid probe by the cleaving agent to release a 5′ flap from the first nucleic acid probe;
b) ligating the 5′ flap to a nucleic acid adaptor to form an adaptor-ligated product, wherein the nucleic acid adaptor comprises a double-stranded region and a 3′overhang extension that is complementary to the 5′ flap and wherein the 3′ overhang extension hybridizes to the 5′ flap;
c) contacting the adaptor-ligated product with:
   i) a type V CRISPR/Cas effector protein;
   ii) a guide RNA comprising a region that binds to the type V CRISPR/Cas effector protein and a guide sequence that is complementary to a portion of the 5′ flap and a portion of the nucleic acid adaptor that is ligated to and adjacent to the 5′ flap; and
   iii) a single stranded detector DNA; and
d) measuring a detectable signal produced by cleavage of the single stranded detector DNA by the type V CRISPR/Cas effector protein to detect the adaptor-ligated product, thereby detecting the polynucleotide analyte in the sample.

Disclosed herein is a kit for detecting a polynucleotide analyte in a sample, comprising a structure-specific nucleic acid cleaving agent, a nucleic acid ligase and a type V CRISPR/Cas effector protein.

### BRIEF DESCRIPTION OF DRAWINGS

Embodiments of the present invention are hereafter described, by way of non-limiting example only, with reference to the accompanying drawings in which:

FIG. 1 Overall schematic of the working of the flap endonuclease, Taq ligase and CRISPR-Cas12 in the disclosed method.

FIG. 2 Illustration of the workflow for polynucleotide detection using FELICX (flap endonuclease, Taq ligase and CRISPR-Cas for diagnostics (X)). (a) Genetic material is extracted from clinical samples and amplified isothermally. (b) A target nucleotide sequence is detected and a signal generated through the sequential activity of FEN, ligase and Cas12-sgRNA. The output of FEN is an oligo, which is ligated to an adaptor to form the substrate for Cas12-sgRNA, binding of the substrate to Cas12 leads to the cleavage of a ssDNA reporter. (c) The output can be a fluorescence signal or a readout on lateral flow strips.

FIG. 3 Binding of the invading primer and flap probe to a DNA target. The underlined sequence is the flap that remains constant for any target. The red arrow represents the point where FEN cleaves the flap probe.

FIG. 4 Flap endonuclease activity of the commonly used Tth pol and Tth pol v2 with DNA as the target. The activity was determined using a flap probe labelled with a fluorophore and a quencher. After cleavage by the purified enzymes, a fluorescent signal is observed. The reaction was carried out in a buffer comprising of Tris-Cl, NaCl, MgCl₂, Triton-X-100 and DTT.

FIG. 5 Ligation of the flap to the dsDNA adaptor comprising a 5′ p adaptor strand and ds converter strand. The flap product is shaded. The box on the top strand of the ligated product is the binding region of the gRNA, and the box on the bottom strand is the PAM sequence for CRISPR-Cas12.

FIG. 6 Detection of (a) orflab copies amplified with LAMP; (b) E. coli genome copies amplified with LAMP; (c) orflab copies amplified with HAD; (d) E. coli genome copies amplified with HDA. NTC-no template control. All the samples were prepared by spiking the target DNA into HEK293t total DNA to simulate clinical samples.

FIG. 7 Lateral flow readout of orflab (simulated samples) after HDA and detection using the method described here. (a) Readout after 1 hr of HDA, 1 hr of FEN+Taq reaction and 30 min of cas12 reaction (b) Readout after 20 min of HDA, 20 min of FEN+Taq reaction and 20 min of cas12 reaction. The 5′6-FAM-TTATTATTAT-3′Bio ssDNA was used as the reporter for the orflab gene which corresponds to T₁. C-control, T₂-second test line not used in this experiment.

FIG. 8(a) Amplification of orflab RNA using RTx and HDA in a two-step or one-step method. The blue triangle shows increasing volume of RTx mixture added to HDA. (b) Lateral flow readout of orflab RNA (simulated sample) in 90 min (45 min of HDA, 20 min of FEN+Taq reaction and 20 min of cas12 reaction. The 5′6-FAM-TTATTATTAT-3′Bio ssDNA was used as the reporter for the orflab RNA which corresponds to T1. C-control, T2-second test line not used in this experiment. NTC-no template control

FIG. 9 Detection of EBV and gapdh in C666-1 and HK-1 cells. NTC-no template control. The graph shows the fluorescence readout from the Cas12 step of the detection method.

FIG. 10 Detection of blaKPC and khe in Klebseilla pneumoniae spiked into HK-1 cells. NTC-no template control. The graph shows the fluorescence readout from the Cas12 step of the detection method.

FIG. 11 Detection of scgb2a2 spiked into FBS. The graph shows the fluorescence readout from the Cas12 step of the detection method.

FIG. 12 Detection of WT or mut DNA corresponding to spike protein of SARS-COV-2 by (a) the wild-type flap probe (b) the mut flap probe. The graph shows the fluorescence readout from the Cas12 step of the detection method.

FIG. 13 shows a comparison of FEN activity of different enzymes and their engineered variants. (a) Schematic of the fluorescence assay for quantifying FEN activity. (b) The activity of different FEN enzymes on 1 nM orflab DNA/RNA substrate with different configurations of the flap and invading primers, including the configuration shown in (a) (first column), a double flap configuration with flaps on both invading and flap primers (second column), a configuration without the invading primer (third column), and a configuration with the flap primer only (fourth column). No FEN refers to the reaction without the enzyme (fifth column). Data on the left is for DNA substrate and data on the right for RNA substrate. The data represent the mean (n=3 biological repeats). Thermo FEN was obtained from NEB (Catalog No. M0645S). (c) FEN activity of Tth pol v2 on the probe (flap primer only), on DNA substrate (1 nM) and on RNA substrate (1 nM) at different enzyme concentrations after 60 min of incubation at 65° C. The horizontal black bars represent the mean (n=3 biological repeats). The p value<0.05 are provided, calculated using a two-sample t-test. (d) and (e) Detection limits of orflab DNA (left) and RNA (right) after incubation with 10 nM Tth pol v2 for 30 min at 65° C. The data represents the mean±SD (n=3 biological repeats). The p value<0.05 compared to NTC are provided, calculated using a two-sample t-test. NTC: no target control.

FIG. 14 shows the optimization of adaptor ligation and nucleic acid detection limit using FELICX. (a) Sequence of a flap product ligated to the dsDNA adaptor. The PAM is underlined and the fickle base pair is shaded. (b) Schematic showing ligation of an oligo, similar to the flap product formed by FEN, to the dsDNA adaptor by a ligase. The ligated product forms the substrate for Cas12-sgRNA which, upon activation, cleaves a ssDNA reporter, resulting in the production of a fluorescence signal. (c) Fluorescence signal observed after ligating the synthetic flap oligo with the dsDNA adaptor for 30 min using different ligases and subsequent detection by Cas12-sgRNA (10 min or 30 min of cleavage). The ligation volume was 15 μL, to which 5 μL of Cas12-sgRNA was subsequently added. The data represents the mean±SD (n=3 biological repeats). NTC: no target control. Ligated: pre-ligated substrate. (d) Optimization of the FEN concentration and incubation time for the FEN+Taq ligase reaction (15 μL) using orflab DNA (1 nM). After ligation, the Cas12-sgRNA reaction (20 μL) was performed for 30 min. The data represents the mean±SD (n=2 biological repeats). (e) and (f) Detection limit of orflab DNA or RNA spiked into purified HEK293T total DNA or RNA, respectively, using present detection system. The FEN+Taq ligase reaction (20) μL) was performed for 4 h and the Cas12-sgRNA reaction (an additional 7 μL) was performed for 30 min. The data represents the mean±SD (n=3 biological repeats). The p value<0.05 compared to NTC are provided, calculated using a two-sample t-test. (g) and (h) Detection limit of HDA+FELICX for the orflab DNA and E. coli genome spiked into HEK293T total DNA. HDA was performed for 1 h in a 10 μL reaction, FEN+Taq ligase reaction for 1 h (total volume 20 μL), and Cas12-sgRNA reaction for 30 min (total volume 27 μL). The data represents the mean±SD (n=3 biological repeats). The p value<0.05 compared to NTC are provided, calculated using a two-sample t-test.

FIG. 15 shows the combination of FELICX with lateral flow strips for nucleic acid detection. (a) Schematic of the lateral flow strip used in this study. Test line T1 corresponds to the reporter with FAM and biotin, while T2 corresponds to the reporter with digoxigenin (DIG) and biotin. C is the lateral flow control. (b) Schematic of nucleic acid detection using HDA+FELICX along with lateral flow strips. Lateral flow output in the presence and absence of the target in the sample is depicted. (c) Lateral flow for the detection of orflab DNA spiked into HEK293T total DNA. HDA was performed for 1 h in a 25 μL reaction followed by rapid purification, FEN+Taq reaction for 1 h (total volume 20 μL), and Cas12-sgRNA reaction for 30 min (total volume 27 μL). In this experiment, only T1 was used. The numbers below the strips show the fold change in the T1 band intensity compared to NTC. (d) Same as (c) with the HDA, FEN+Taq, and Cas12-sgRNA reactions performed for 20 min each. The numbers below the strips show the fold change in the T1 band intensity compared to NTC. (e) Lateral flow for the detection of orflab RNA spiked into HEK293T total RNA. HDA was performed for 45 min in a 25 μL reaction followed by rapid purification, FEN+Taq reaction for 20 min (total volume 20 μL), and Cas12-sgRNA reaction for 20 min (total volume 27 μL). Only T1 was used in the experiment. The numbers below the strips show the fold change in the T1 band intensity compared to NTC.

FIG. 16 shows SNP detection using FELICX. (a) Schematic showing the cleavage of the flap primer by the FEN in the case of the wild-type (WT) target, and no flap primer cleavage in the case of a SNP. The cleavage site of the flap primer is marked by the arrow: The last nucleotide of the invading primer is mismatched with the target, which prevents FEN cleavage. (b) Detection of the WT target, target with SNP/mutation or no target with FELICX and the corresponding lateral flow strip pattern observed in each case. (c)-(e) Detection of the WT receptor binding domain sequence of SARS-CoV-2 or its variant (T478K) by FELICX using WT and mut probes. Panel (c) shows the fluorescence signals, whereas (d) shows the lateral flow result after FELICX was performed on pre-amplified substrates using both probes. Pre-amplification was performed through PCR, followed by DNA purification, to ensure that an equal mass of both WT and mutated DNA was used for accurate comparison. The FEN+Taq reaction was performed for 60 min (total volume 20 μL), and the Cas12-sgRNA reaction was performed for 30 min (total volume 27 μL). In (c), the data represents the mean±SD (n=3 biological repeats). The p value<0.05 compared to NTC are provided, calculated using a two-sample t-test. (e) Percentage change in band intensity corresponding to WT and mut probes compared to NTC in (d). NTC: no target control.

FIG. 17 is a demonstration of the versatility of FELICX. FELICX was used to detect targets in more complex samples, such as whole bacterial and mammalian cells and serum. Fold change in the fluorescence signal for the detection of (a) EBV and gapdh in whole C666-1 and HK-1 cells, (b) bla_{KPC} and khe in K. pneumoniae (Kp) cells spiked into HK-1 whole cells and (c) SCGB2A2 RNA spiked into FBS. Samples were lysed and rapidly purified as described in the Methods. HDA was performed for 1 h in a 10 μL reaction, FEN+Taq ligase reaction for 1 h (total volume 20 μL), and Cas12-sgRNA reaction for 30 min (total volume 27 μL). NTC: no target control.

FIG. 18. (a) Schematic showing the various FEN enzymes used in this study. The boxes denote the domains of the enzyme and the lines represent the positions of point mutations introduced in the enzyme. (b) Table showing the point mutations introduced in Tth pol v1, v2 and v3. (c) SDS-PAGE showing the purification of different FEN enzymes. Proteins of interest are labeled with the arrowheads. (d) Schematic of the fluorescence assay used to quantify FEN activity. This configuration shows a 5′-flap for the flap primer and 3′-flap for the invading primer.

FIG. 19. (a) Structure of the T5 exonuclease (PDB ID: 1EXN) with the helical arch labeled. (b) Predicted structure of Tth pol with the disordered arch labeled. The structure was predicted using SWISS-MODEL5. (c) FEN activity of different enzymes with 1 nM DNA (left panel) and 1 nM RNA (right panel) as the substrate. The data represents the mean±SD (n=3 biological repeats).

FIG. 20. (a) and (b) Detection limit of orflab DNA and RNA, respectively, after incubation of 50 nM Tth pol v2 for 30 min at 65° C. The data represents the mean±SD (n=3 biological repeats). The p values<0.05 compared to the NTC are provided, calculated using a two-sample t test. NTC: no target control.

FIG. 21 shows Cas12-sgRNA activity with either ssDNA (1 nM) or dsDNA (1 nM) as the substrate, determined by co-incubation at 37° C. in a 20 μL reaction and measuring fluorescence signals generated by reporter cleavage. The data represents the mean (n=3 biological repeats).

FIG. 22 shows fluorescence signals observed upon varying the Cas12-sgRNA complex (RNP) and its DNA reporter (rep) using dsDNA substrate (1 nM) after 30 min incubation at 37° C. RNP (1×)=120 nM of Cas12 and 100 nM of sgRNA, RNP (2×)=200 nM of Cas12 and 167 nM of sgRNA, rep (1×)=80 nM of DNA reporter and rep (2×)=133.3 nM of DNA reporter. NTC: no target control. The data represents mean±SD (n=2 biological repeats).

FIGS. 23(a) and (b) Schematic of the pre-ligated dsDNA adaptor with N=A, C, G or T (shaded box) hybridized to sgRNA and sgRNA-7DNA, respectively. Both gRNAs are complementary to the dsDNA adaptor with N=T. The PAM sequence is underlined. (c) Activity of the Cas12-sgRNA and Cas12-sgRNA-7DNA complexes on four possible pre-ligated dsDNA adaptors (5 nM) determined by co-incubation at 37° C. in a 20 μL reaction and measuring fluorescence signals generated by reporter cleavage. The data represents the mean (n=3 biological repeats). NTC: no target control.

FIGS. 24(a) and (b) Detection limit of orflab DNA or E. coli genome spiked into purified HEK293tT total DNA using LAMP+FELICX. The LAMP reaction was performed for 30 min in a 10 μL reaction, FEN+Taq reaction for 1 hr (total volume 20 μL) and Cas12-sgRNA reaction for 30 min (total volume 27 μL). The data represents the mean±SD (n=3 biological repeats). The p values<0.05 compared to the NTC are provided, calculated using a two-sample t test. NTC: no target control.

FIG. 25 shows Cq values for LAMP performed using different primer sets following the manufacturer's instructions at 65° C. DNA: 60,000 copies of orflab DNA. NTC: no target control. For set 1, a biological repeat of the NTC did not show any amplification and hence, no Cq value was determined. Horizontal black bars represent the mean (n=3 biological repeats).

FIG. 26(a) Agarose gel showing the amplification of orflab RNA by two-step or one-step RTx+HDA. In the two-step method, reverse transcription was performed using RTx for 10 min, followed by HDA for 50 min at 65° C. In the one-step method, RTx+HDA were simultaneously performed by incubation for 60 min at 65° C. No amplification was observed for NTC (no target control) samples. (b) Quantification of the band intensity corresponding to the amplified product as seen in (a). Quantification was performed using ImageJ. The data represents the mean±SD (n=3 biological repeats).

FIG. 27 shows the fold-change in fluorescence signal for the detection of EBV and gapdh in C666-1 and HK-1 cell lines (top panel), and bla_{KPC} and khe in K. pneumoniae (Kp) spiked into HK-1 cells (bottom panel). Samples were lysed and rapidly purified as described in the Methods. HDA was performed for 1 hr in a 10 μL reaction, FEN+Taq ligase reaction for 1 hr (total volume 20 μL), and Cas12-sgRNA reaction for 30 min (total volume 27 μL). NTC: no target control.

### DETAILED DESCRIPTION

The present specification teaches a method of detecting a polynucleotide analyte in a sample.

The method may comprise providing a first nucleic acid probe and a second nucleic acid probe that are configured to form a cleavage structure in the presence of the polynucleotide analyte. The method may comprise a) contacting the sample comprising the polynucleotide analyte with: i) a first nucleic acid probe comprising a 3′ portion complementary to a first portion of the polynucleotide analyte and a 5′ portion that is not complementary to and does not hybridize to the polynucleotide analyte: ii) a second nucleic acid probe comprising a 5′ portion complementary to a second portion of said polynucleotide analyte and a 3′ portion that is not complementary to and does not hybridize to the polynucleotide analyte, wherein said first portion of the polynucleotide analyte is 5′ to and contiguous with the second portion of the polynucleotide analyte; and iii) a structure-specific nucleic acid cleaving agent: wherein hybridization of the first nucleic acid probe to the first portion of the polynucleotide analyte and hybridization of the second nucleic acid probe to the second portion of the polynucleotide analyte forms a cleavage structure: wherein formation of the cleavage structure allows cleavage of the first nucleic acid probe by the cleaving agent to release a 5′ flap comprising the 5′ portion of the first nucleic acid probe that is not complementary to and does not hybridize to the polynucleotide analyte. The method may further comprise b) ligating the 5′ flap to a nucleic acid adaptor to form an adaptor-ligated product, wherein the nucleic acid adaptor comprises a double-stranded region and a 3′overhang extension that is complementary to the 5′ flap and wherein the 3′ overhang extension hybridizes to the 5′ flap to the nucleic acid adaptor. The method may further comprise c) contacting the adaptor-ligated product with: i) a type V CRISPR/Cas effector protein: ii) a guide RNA comprising a region that binds to the type V CRISPR/Cas effector protein and a guide sequence that is complementary to a portion of the 5′ flap and a portion of the nucleic acid adaptor that is ligated to and adjacent to the 5′ flap; and iii) a single stranded detector DNA; and d) measuring a detectable signal produced by cleavage of the single stranded detector DNA by the type V CRISPR/Cas effector protein to detect the adaptor-ligated product, thereby detecting the polynucleotide analyte in the sample.

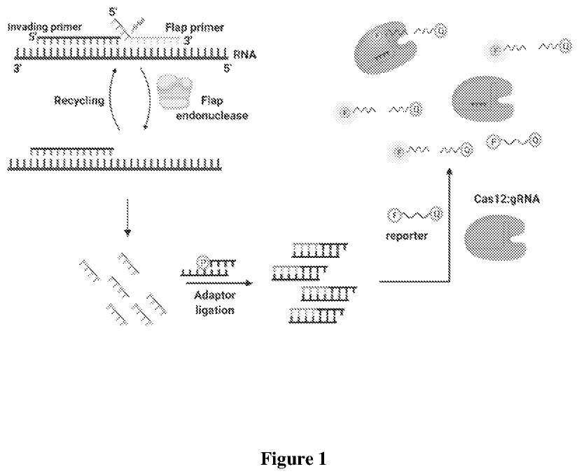

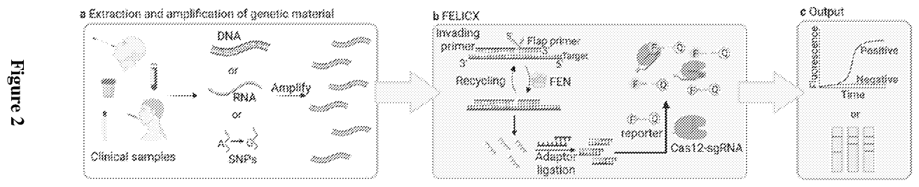

Without being bound by theory, the invention incudes the use of a structure-specific nucleic acid cleaving agent (e.g., a flap endonuclease), a nucleic acid ligase and a Type V CRISPR-Cas nuclease to detect nucleic acids (either DNA or RNA) rapidly with high sensitivity and specificity. The sample is first incubated with a pair of DNA oligonucleotides that bind to the target DNA/RNA to create a flap structure which is recognized by the structure-specific nucleic acid cleaving agent and which is subsequently cleaved off (FIG. 1). The cleaved flap product is ligated to a double-stranded DNA (dsDNA) adaptor by a nucleic acid ligase to form a substrate for the CRISPR-Cas nuclease. In the presence of the substrate, the Cas nuclease, bound to its guide RNA (gRNA), cleaves a single-stranded DNA (ssDNA) reporter to generate a detectable signal, which may be, e.g., a visual band on a lateral flow detection system, or a fluorescent signal. Different nucleic acids, even those differing by a single nucleotide, can be distinguished by using DNA oligonucleotides and adaptors specific to each nucleic acid. The sensitivity of the method can be increased by incorporating an isothermal amplification system, such as loop-mediated isothermal amplification (LAMP) or helicase-dependent amplification (HDA), before DNA oligonucleotide binding, and using the product of this amplification as the substrate for the series of binding and enzymatic reactions of this disclosure.

As used herein, the term “nucleic acid”, and equivalent terms such as “polynucleotide”, refer to a polymeric form of nucleotides of any length, such as ribonucleotides, deoxyribonucleotides or peptide nucleic acids (PNAs), that comprise purine and pyrimidine bases, or other natural, chemically or biochemically modified, non-natural, or derivatized nucleotide bases. The nucleic acid may be double-stranded or single-stranded. References to single-stranded nucleic acids include references to the sense or antisense strands. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The terms nucleoside, nucleotide, deoxynucleoside and deoxynucleotide generally include complements, fragments and variants of the nucleoside, nucleotide, deoxynucleoside and deoxynucleotide, or analogs thereof.

An “oligonucleotide” as used herein is a single stranded molecule which may be used in hybridization or amplification technologies. In general, an oligonucleotide may be any integer from about 15 to about 100 nucleotides in length, but may also be of greater length.

The term “complementary” refers to the base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100% of the nucleotides of the other strand.

The term “not complementary” or “non-complementary” may refer to the lack of base pairing between nucleotides or nucleic acids, such as, for instance, between the two strands of a double stranded DNA molecule or between an oligonucleotide primer and a primer binding site on a single stranded nucleic acid to be sequenced or amplified. Two single stranded RNA or DNA molecules may be considered to be not complementary when the nucleotides of one strand pair with less than 50% of the nucleotides of the other strand, optionally less than 40%, 30%, 20% or 10% of the nucleotides of the other strand.

As used herein, the term “hybridization” or “hybridizes” refers to the process in which two single-stranded polynucleotides bind non-covalently to form a stable double-stranded polynucleotide, i.e., a duplex. The term “hybridization” may also refer to triple-stranded hybridization. The resulting (usually) double-stranded polynucleotide is a “hybrid”. The proportion of the population of polynucleotides that forms stable hybrids is referred to herein as the “degree of hybridization”.

Hybridization conditions will typically include salt concentrations of less than about IM, more usually less than about 500 mM and less than about 200 mM. Hybridization temperatures are typically greater than 22° C., more typically greater than about 30° C., and preferably in excess of about 37° C. In one embodiment, hybridization takes place at about 65° C. Hybridizations are usually performed under stringent conditions, i.e., conditions under which a probe will hybridize to its target. Stringent conditions are sequence-dependent and are different under different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point (Tₘ) for the specific sequence at a defined ionic strength and pH. The Tₘ is the temperature (under defined ionic strength, pH and nucleic acid composition) at which 50% of the probes complementary to the target sequence hybridize to the target sequence at equilibrium.

The term “probe” refers to any molecule which is capable of selectively binding to a specifically intended target nucleic acid, for example, genomic DNA, a polynucleotide transcript, viral DNA or RNA. Probes can either be synthesized by one skilled in the art, or derived from appropriate biological preparations.

As used herein, the term “cleavage structure” refers to a structure that is formed by the interaction of at least one nucleic acid probe and a target polynucleotide, the structure comprising a duplex with an adjacent single-stranded nucleic acid flap, this structure being cleavable by a structure-specific nucleic acid cleaving agent. The secondary structure of the cleavage structure makes it a suitable substrate for specific cleavage by the structure-specific nucleic acid cleaving agent.

As used herein, the term “flap probe” or “flap oligonucleotide” refers to an oligonucleotide that interacts with a target polynucleotide to form a cleavage structure, whether in the presence or absence of an invader oligonucleotide. When hybridized to the target polynucleotide, the flap probe and target form a cleavage structure and cleavage occurs within the flap probe.

As used herein, the term “invader probe” or “invader oligonucleotide” refers to an oligonucleotide that hybridizes to a target polynucleotide at a location near the region of hybridization between a flap probe and the target nucleic acid, wherein the invader probe comprises a portion (e.g., a chemical moiety or nucleotide-whether complementary to that target or not) that overlaps with the region of hybridization between the flap probe and target.

The first nucleic acid probe (or flap probe) of the present disclosure may comprise a 3′ portion complementary to a first portion of the polynucleotide analyte. This 3′ portion may comprise or consist of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleobases. The first nucleic acid probe may comprise a 5′ portion that is not complementary to and does not hybridize to the polynucleotide analyte. This 5′ portion may be 5, 6, 7, 8, 9, 10, 11, 12, 13, 14, 15, 16, 17, 18, 19, 20, 21, 22, 23, 24, 25 or more nucleobases.

The second nucleic acid probe (or invader probe) of the present disclosure may comprise a 5′ portion complementary to a second portion of said polynucleotide analyte and a 3′ portion that is not complementary to and does not hybridize to the polynucleotide analyte. This 5′ portion may comprise or consist of about 20 to 50 nucleobases. The 5′ portion may comprise or consist of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleobases. The 3′ portion may comprise or consist of 1, 2, 3, 4, 5, 6, 7, 8, 9, 10, 11, 12, 13, 14, 15, 16, 17, 18, 19, 20, 21, 22, 23, 24 or 25 nucleobases. In one embodiment, the 3′ portion consists of 1 nucleobase.

In one embodiment, the first nucleic acid probe and second nucleic acid probe overlap with each other by at least one nucleobase when hybridized to the polynucleotide analyte. The first nucleic acid probe and second nucleic acid probe may overlap with each other by 1, 2, 3, 4, 5, 6, 7, 8, 9, 10, 11, 12, 13, 14, 15, 16, 17, 18, 19, 20, 21, 22, 23, 24 or 25 nucleobases.

In one embodiment the cleavage structure is formed from hybridization of the first and second nucleic acid probes to the target polynucleotide. This cleavage structure may comprise a duplex formed between the first nucleic acid probe and the polynucleotide, and a 5′ flap comprising the 5′ portion of the first nucleic acid probe that does not hybridize to the polynucleotide. This cleavage structure may further comprise a 3′ flap comprising the 3′ portion of the second nucleic acid probe that does not hybridize to the polynucleotide. The 5′ flap may be 1, 2, 3, 4, 5, 6, 7, 8, 9, 10, 11, 12, 13, 14, 15, 16, 17, 18, 19, 20, 21, 22, 23, 24, 25 or more nucleotides in length. The 3′ flap may be 1, 2, 3, 4 or 5 nucleotides in length.

As used herein the term “structure-specific nucleic acid cleaving agent” or “cleaving agent” refers to any agent that is capable of cleaving a cleavage structure, including but not limited to enzymes. “Structure-specific nucleases” or “structure-specific enzymes” are enzymes that recognize specific secondary structures in a nucleic acid molecule and cleave these structures. A structure-specific nucleic acid cleaving agent of this disclosure cleaves a nucleic acid molecule in response to the formation of cleavage structures: it is not necessary that the cleaving agent cleave the cleavage structure at any particular location within the cleavage structure. The cleaving agent may include nuclease activity provided from a variety of sources including flap endonucleases (FEN) from prokaryotes and eukaryotes, DNA polymerases (e.g., Taq DNA polymerase, DNA polymerase from Thermus sp. and E. coli DNA polymerase I) and exonucleases (e.g., bacteriophage T5 exonuclease). The cleaving agent may include enzymes having 5′ nuclease activity, e.g., Taq DNA polymerase, E. coli DNA polymerase I, bacteriophage T5 exonuclease, Thermus sp. DNA polymerase. The cleaving agent may also include modified or engineered DNA polymerases having 5′ nuclease activity but lacking synthesis activity or exhibiting reduced synthesis activity.

As used herein the term “cleavage product” or “5′ cleavage product” refers to a product generated by the action of a structure-specific nucleic acid cleaving agent on a cleavage structure, e.g., a 5′ flap cleaved from a flap probe.

In one embodiment, the formation of the cleavage structure allows cleavage of the first nucleic acid probe by the cleaving agent to release a 5′ flap comprising the 5′ portion of the first nucleic acid probe that is not complementary to and does not hybridize to the polynucleotide analyte.

The cleaving agent may cleave the first nucleic acid probe at a position one nucleobase 3′ of the portion of the first nucleic acid probe that overlaps with the second nucleic acid probe.

In some embodiments the structure-specific nucleic acid cleaving agent is an enzyme with flap endonuclease activity.

As used herein, the term “flap endonuclease (FEN)” refers to an enzyme with 5′ exonuclease and structure-specific endonuclease activity which recognizes and cleaves a cleavage structure to generate a 5′ cleavage product. Enzymes with FEN activity herein may be wild-type or engineered enzymes from prokaryotes or eukaryotes, e.g., flap endonucleases from the FEN1 family of proteins, bacterial DNA polymerases, bacteriophage T5 and T7 exonucleases, and nucleases from the XPG/Rad2 superfamily. Such enzymes may comprise one or more additional enzymatic activities in addition to FEN activity, e.g., nucleic acid polymerase activity. The enzymes may be engineered to reduce or remove non-FEN activity.

In some embodiments the enzyme with FEN activity is a thermostable enzyme.

The term “thermostable” when used in reference to an enzyme, such as an enzyme with FEN activity or a nucleic acid ligase, indicates that the enzyme is functional or active (i.e., can perform catalysis) at an elevated temperature, e.g., at 45° C. or higher. In some embodiments a thermostable enzyme herein is functional at temperatures of between about 45° C. to about 80° C., about 50° C. to about 75° C., about 55° C. to about 70° C., or between about 60° C. to about 70° C. In some embodiments a thermostable enzyme is functional at a temperature of 45° C. or higher, 50° C. or higher, 55° C. or higher, 60° C. or higher, 65° C. or higher, 70° C. or higher, or 75° C. or higher. In one embodiment, a thermostable enzyme herein is functional at a temperature of about 45° C. to about 80° C.

In some embodiments the enzyme is a DNA polymerase from Thermus thermophiles (i.e., a Tth polymerase).

The enzyme may be a wild-type, mutant isoform or genetically engineered Tth polymerase. Wild-type Tth polymerase possesses both FEN activity and polymerase activity. Tth polymerases for use in the methods herein may have reduced or negligible polymerase activity.

An engineered Tth polymerase may comprise one or more amino acid substitutions, insertions or deletions. The term “deletion”, used in relation to an amino acid, means that the amino acid has been removed or is absent. The term “insertion” means that one or more amino acids have been added. A “substitution” means that an amino acid residue is replaced by another amino acid residue. An amino acid residue may be substituted with another selected from the naturally-occurring standard 20 amino acid residues, rare naturally occurring amino acid residues (e.g. hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g. cyclohexyl-alanine). Preferably, the term “substitution” refers to the replacement of an amino acid residue by another selected from the naturally-occurring standard 20 amino acid residues (G, P, A, V, L, I, M, C, F, Y, W, H, K, R, Q, N, E, D, S and T).

A “conservative amino acid substitution” is one in which the amino acid residue is replaced with an amino acid residue having a similar side chain. Families of amino acid residues having similar side chains have been defined in the art, which can be generally sub-classified as follows:

### a) Amino Acid Sub-Classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine |
| Residues that influence | Glycine and Proline |
| chain orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity. Conservative substitutions are shown in the table below under the heading of exemplary substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

### b) Exemplary Amino Acid Substitutions

| Original Residue | Exemplary Substitutions |
|---|---|
| Ala | Val, Leu, Ile |
| Arg | Lys, Gln, Asn |
| Asn | Gln, His, Lys, Arg |
| Asp | Glu |
| Cys | Ser |
| Gln | Asn, His, Lys, |
| Glu | Asp, Lys |
| Gly | Pro |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Ile, Phe |
| Phe | Leu, Val, Ile, Ala |
| Pro | Gly |
| Ser | Thr |
| Thr | Ser |
| Trp | Tyr |
| Tyr | Trp, Phe, Thr, Ser |
| Val | Ile, Leu, Met, Phe, Ala |

In some embodiments the enzyme with FEN activity is a Thermus thermophiles DNA polymerase comprising one or more amino acid substitutions. In some embodiments the one or more amino acid substitutions is selected from the group consisting of: A599T, A604V, W606R, A607Q, A610V, I616M, 1616T and E617G. In some embodiments the Thermus thermophiles DNA polymerase comprises the following amino acid substitutions: A604V, A610V, I616M and E617G. In some embodiments the Thermus thermophiles DNA polymerase comprises the following amino acid substitutions: A599T, W606R, A607Q and I616T.

An engineered Tth polymerase which recognizes both DNA and RNA substrates was previously described in WO2001090337A2. The inventors have further engineered this Tth polymerase variant by introducing mutations at positions 604, 610, 616 and 617 (i.e., A604V, A610V, I616M and E617G) to give the enzyme with amino acid sequence given in SEQ ID NO: 1. The inventors have also introduced mutations at positions 599, 606, 607 and 616 (i.e., A599T, W606R, A607Q and 1616T) to give the enzyme with amino acid sequence given in SEQ ID NO: 2. These enzymes exhibit higher FEN activity for a given substrate (i.e., a given cleavage structure) and lower non-specific activity towards oligonucleotides which do not form a cleavage structure.

In some embodiments the enzyme comprises an amino acid sequence having at least 80% sequence identity to an amino acid sequence set forth in SEQ ID NO: 1 or SEQ ID NO: 2. In one embodiment the enzyme comprises an amino acid sequence having at least 80% sequence identity to an amino acid sequence set forth in SEQ ID NO: 1. In one embodiment the enzyme comprises an amino acid sequence having at least 80% sequence identity to an amino acid sequence set forth in SEQ ID NO: 2.

In some embodiments step (a) of the methods herein is performed isothermally, i.e., the temperature is unchanged for the duration of step (a). Step (a) may be performed at a temperature of 40° C. or higher. In some embodiments step (a) is performed at a temperature of between about 40° C. to about 90° C., between about 50° C. to about 80° C., between about 55° C. to about 75° C., or between about 60° C. to about 70° C. In one embodiment, step (a) is performed isothermally at a temperature of about 65° C.

The term “adaptor” refers to a nucleic acid molecule that is configured to be complementary to both a cleavage product of the structure-specific cleaving agent (such as a 5′ flap) and to a guide RNA of a type V CRISPR/Cas effector protein (e.g., Cas12). The “adaptor” may comprise a 3′ overhang that is complementary to the 5′ flap. The “adaptor” is advantageous in the present methods as it bridges steps (a) and (c) by enabling the Cas nuclease in step (c) to recognize the cleavage product of step (a).

In some embodiments the nucleic acid adaptor comprises a first adaptor oligonucleotide comprising a protospacer-adjacent motif (PAM) of a type V CRISPR/Cas effector protein, and a second adaptor oligonucleotide that is complementary to and hybridized to the first adaptor oligonucleotide. The first adaptor oligonucleotide may be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleobases. The second adaptor oligonucleotide may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or more nucleobases.

In some embodiments the first adaptor oligonucleotide comprises a PAM sequence of a Cas12 nuclease. In some embodiments the PAM sequence is 5′-TTTN-3′, wherein N may be any one of A, T, C or G.

In some embodiments the second adaptor oligonucleotide comprises a 5′ modification, e.g., a modification to a nucleobase or to the sugar-phosphate backbone. The 5′ modification may permit action by a subsequent enzyme in the methods herein. In one embodiment the second adaptor oligonucleotide is phosphorylated at its 5′ end to prime it for subsequent ligation by a nucleic acid ligase.

In some embodiments step (c) is performed in the presence of a nucleic acid ligase. The 5′ cleavage product of the cleaving agent may be ligated to the adaptor by the ligase following hybridization with the 3′ overhang of the nucleic acid adaptor. The term “adaptor-ligated product”, as used herein, refers to a nucleic acid (such as a 5′ flap) that has been ligated to an adaptor.

Non-limiting examples of enzymes that can be used for ligation in the methods disclosed herein are ATP-dependent double-stranded polynucleotide ligases, NAD+ dependent DNA or RNA ligases, and single-strand polynucleotide ligases. Non-limiting examples of ligases are E. coli DNA ligase, Thermus filiformis DNA ligase, Tth DNA ligase, Thermus scotoductus DNA ligase (I and II), T3 DNA ligase, T4 DNA ligase, T4 RNA ligase, T7 DNA ligase, Taq ligase, VanC-type ligase, 9°N DNA ligase, Tsp DNA ligase, DNA ligase I, DNA ligase III, DNA ligase IV, Sso7-T3 DNA ligase, Sso7-T4 DNA ligase, Sso7-T7 DNA ligase, Sso7-Taq DNA ligase, Sso7-E. coli DNA ligase, and Sso7-Ampligase DNA ligase. Ligases herein may be wild-type, mutant isoforms, or genetically engineered variants.

In some embodiments the ligase is a thermostable ligase, e.g., Taq ligase.

In some embodiments step (b) of the methods herein is performed isothermally, i.e., the temperature is unchanged for the duration of step (b). Step (b) may be performed at a temperature of 40° C. or higher. In some embodiments step (b) is performed at a temperature of between about 40° C. to about 90° C., between about 50° C. to about 80° C., between about 55° C. to about 75° C., or between about 60° C. to about 70° C. In one embodiment, step (b) is performed isothermally at a temperature of about 65° C.

The method can include a CRISPR/Cas effector protein or enzyme and guide RNA. CRISPR/Cas systems, including CRISPR-Cas12 and CRISPR-Cas13, exhibit robust collateral activity against single-stranded DNA (ssDNA) and ssRNA targets, respectively. The collateral cleavage of a non-specific target following recognition and cleavage of the specific target by the Cas effector protein (complexed with guide RNA) provides the basis for highly specific, sensitive approaches for nucleic acid detection.

In some embodiments, the CRISPR/Cas effector is a DNA editing enzyme (e.g., DNA endonuclease) with dsDNA cleavage activity and ssDNA cleavage activity. In such embodiments, the CRISPR/Cas effector can be a class II, type V CRISPR/Cas effector, such as a Cas12 effector protein. Exemplary Cas12 effector proteins include Cas12a, Cas12b, Cas12c, Cas12d, Cas12e, C2c4, C2c8, C2c5, C2cl0, and C2c9.

In some embodiments, the type V CRISPR/Cas effector protein is a Cas12 protein. In one embodiment, the CRISPR/Cas effector protein is Cas12a.

The Cas effector protein variant may include one or more mutations (e.g., conservative or non-conservative mutations). For example, it is also contemplated that other Cas12 variants can be evolved from those disclosed herein, for example, by targeted mutation of one or more amino acid residues in specific regions of the enzyme. Such mutation(s) may alter substrate binding, alter conformation of bound substrate, alter substrate accessibility to the active site, alter tolerance to non-optimal presentation of a target sequence to the active site, and/or alter target sequence specificity (recognition).

A nucleic acid molecule (e.g., a natural crRNA) that binds to a type V CRISPR/Cas effector protein (e.g., a Cas12 protein such as Cas12a, Cas12b, Cas12c, Cas12d, Cas12e), forming a ribonucleoprotein complex (RNP), and targets the complex to a specific target sequence within a target DNA is referred to herein as a “guide RNA”. It is to be understood that in some cases, a hybrid DNA/RNA can be made such that a guide RNA includes DNA bases in addition to RNA bases—but the term “guide RNA” is still used herein to encompass such hybrid molecules.

A guide RNA herein comprises a guide sequence (also referred to as a “spacer”) that hybridizes to a portion of the 5′ flap and a portion of the nucleic acid adaptor. This ensures that the Cas nuclease as active only after a 5′ cleavage product is generated and hybridized to a nucleic acid adaptor. The guide RNA further comprises a constant region (e.g., a region that is adjacent to the guide sequence and binds to the type V CRISPR/Cas effector protein).

The guide sequence may be 15, 16, 17, 18, 19, 20, 21, 22, 23, 24, 25, 26, 27, 28, 29, 30, 31, 32, 33, 34 or 35 or more nucleobases. The guide RNA may be 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more nucleobases.

In some embodiments step (c) of the methods herein is performed isothermally, i.e., the temperature is unchanged for the duration of step (c). Step (c) may be performed at a temperature of 20° C. or higher. In some embodiments step (c) is performed at a temperature of between about 20° C. to about 70° C., between about 25° C. to about 60° C., between about 30° C. to about 50° C., or between about 35° C. to about 40° C. In one embodiment, step (c) is performed at a temperature of between about 20° C. to about 65° C. In one embodiment, step (c) is performed isothermally at a temperature of about 37° C.

The method may comprise cleavage of a single stranded detector DNA (ssDNA) by the type V CRISPR/Cas effector protein to generate a detectable signal.

The ssDNA as described herein may be 7, 8, 9, 10, 11, 12, 13, 14, 15, 16, 17, 18, 19, 20 or more nucleobases. In one embodiment, the ssDNA is AT-rich. In some embodiments, the ssDNA is labelled. The ssDNA may be labeled at the 5′ end, the 3′ end, or at both 3′ and 5′ ends. The ssDNA may also be labeled at an internal position. In one embodiment, the ssDNA is labeled at both ends. The pair of labels may, for example, be FAM and biotin, DIG and biotin, FAM and DIG, or a signal-quencher pair.

In some embodiments the labelled ssDNA comprises a signal-quencher pair. The signal partner of a signal-quencher pair produces a detectable signal and the quencher partner quenches (i.e., reduces) the detectable signal of the signal partner when the signal-quencher partners are in proximity to one another, e.g., when the partners are present on the same ssDNA molecule prior to cleavage by a Type V CRISPR/Cas effector protein. A detectable signal is produced when the labeled ssDNA is cleaved and the signal partner is no longer in proximity to the quencher partner.

A quencher moiety can quench a signal from the signal moiety to various degrees. The quencher moiety may quench the signal from the signal moiety where the signal detected in the presence of the quencher moiety (when the signal partners are in proximity to one another) is 95% or less of the signal detected in the absence of the quencher moiety (when the signal partners are separated). For example, the signal detected in the presence of the quencher moiety can be 90% or less, 80% or less, 70% or less, 60% or less, 50% or less, 40% or less, 30% or less, 20% or less, 15% or less, 10% or less, or 5% or less of the signal detected in the absence of the quencher moiety. No signal (e.g., above background) may be detectable in the presence of the quencher moiety.

The signal detected in the absence of the quencher moiety (when the signal partners are separated) may be at least 1.2 fold greater (e.g., at least 1.3 fold, at least 1.5 fold, at least 1.7 fold, at least 2 fold, at least 2.5 fold, at least 3 fold, at least 3.5 fold, at least 4 fold, at least 5 fold, at least 7 fold, at least 10 fold, at least 20 fold, or at least 50 fold greater) than the signal detected in the presence of the quencher moiety (when the signal partners are in proximity to one another).

In one embodiment, the single stranded detector DNA comprises a fluorophore-quencher pair. The quencher moiety reduces the fluorescent signal from the fluorophore, e.g., by absorbing energy in the emission spectra of the fluorophore. Any convenient fluorophore-quencher pair may be used and many suitable pairs are known in the art.

The quencher moiety may absorb energy from the fluorophore and then emit a signal (e.g., light at a different wavelength). Thus the quencher moiety may itself be a second fluorophore (e.g., a first fluorophore can be 6-carboxyfluorescein while the quencher or second fluorophore can be 6-carboxy-tetramethylrhodamine). The fluorophore-quencher pair could also be a FRET pair. A quencher moiety may be a dark quencher. A dark quencher can absorb excitation energy and dissipate the energy in a different way (e.g., as heat). Thus, a dark quencher has minimal to no fluorescence of its own (does not emit fluorescence).

Examples of fluorophores include, but are not limited to: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), tetramethylrhodamine (TRITC), Texas Red, Oregon Green, Pacific Blue, Pacific Green, Pacific Orange, quantum dots, and a tethered fluorescent protein.

Examples of quencher moieties include, but are not limited to: a dark quencher, a Black Hole Quencher® (BHQ®) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and metal clusters such as gold nanoparticles, and the like.

In one embodiment the single stranded detector DNA comprises a FRET pair. FRET is a process by which radiationless transfer of energy occurs from an excited state fluorophore to a second chromophore in close proximity The range over which the energy transfer can take place is limited to approximately 10 nanometers (100 angstroms), and the efficiency of transfer is extremely sensitive to the separation distance between fluorophores. Thus, as used herein, the term “FRET” (“fluorescence resonance energy transfer”; also known as “Førster resonance energy transfer”) refers to a physical phenomenon involving a donor fluorophore and a matching acceptor fluorophore selected so that the emission spectrum of the donor overlaps the excitation spectrum of the acceptor, and further selected so that when donor and acceptor are in close proximity (usually 10 nm or less) to one another, excitation of the donor will cause excitation of and emission from the acceptor, as some of the energy passes from donor to acceptor via a quantum coupling effect. Thus, a FRET signal serves as a proximity gauge of the donor and acceptor; only when they are in close proximity to one another is a signal generated. The FRET donor moiety (e.g., donor fluorophore) and FRET acceptor moiety (e.g., acceptor fluorophore) are collectively referred to herein as a “FRET pair”.

FRET donor and acceptor moieties (FRET pairs) will be known to one of ordinary skill in the art and any convenient FRET pair (e.g., any convenient donor and acceptor moiety pair) can be used. Examples of FRET pairs are given in the table below.

| Donor | Acceptor |
|---|---|
| Tryptophan | Dansyl |
| IAEDANS | DDPM |
| [5-(2- | [5-(2- |
| iodoacetylaminoethyl)aminonaphthalene- | iodoacetylaminoethyl)aminonaphthalene- |
| 1-sulfonic acid] | 1-sulfonic acid] |
| BFP | DsRFP |
| Dansyl | Fluorescein isothiocyanate (FITC) |
| Dansyl | Octadecylrhodamine |
| Cyan fluorescent | Green fluorescent protein |
| protein (CFP) | (GFP) |
| CF | Texas Red |
| [carboxyfluorescein | |
| succinimidyl ester] | |
| Fluorescein | Tetramethylrhodamine |
| Cy3 | Cy5 |
| GFP | Yellow fluorescent protein (YFP) |
| BODIPY FL (4) | BODIPY FL |
| | [4,4-difluoro-4-bora-3a,4a-diaza-s- |
| | indacene] |
| Rhodamine 110 | Cy3 |
| Rhodamine 6G | Malachite Green |
| FITC | Eosin Thiosemicarbazide |
| B-Phycoerythrin | Cy5 |
| Cy5 | Cy5.5 |

The labeled detector ssDNA may produce a first detectable signal prior to being cleaved (e.g., from a FRET pair) and a second detectable signal when the labeled detector ssDNA is cleaved (e.g., from a quencher/fluorophore pair). As such, the labeled detector ssDNA may comprise a FRET pair and a quencher/fluorophore pair.

In some cases, cleavage of a labeled detector ssDNA can be detected by measuring a colorimetric read-out. For example, the liberation of a fluorophore (e.g., liberation from a FRET pair, liberation from a quencher/fluorophore pair, and the like) can result in a wavelength shift (and thus color shift) of a detectable signal. Thus, in some cases, cleavage of a labeled detector ssDNA herein can be detected by a color-shift. Such a shift can be expressed as a loss of an amount of signal of one color (wavelength), a gain in the amount of another color, a change in the ratio of one color to another, and the like.

In some embodiments the cleavage of the single stranded detector DNA is detected visually. Visual detection may be by direct observation (e.g., by eye or using a camera or a microscope) or via spectroscopic or spectrophotometric measurement.

In one embodiment the cleavage of the single stranded detector DNA is detected by measuring a change in a fluorescent signal produced by the detector DNA. The change in fluorescent signal may be an increase or a decrease in fluorescence when the detector DNA is used in a method herein, e.g., in step (c) of a method herein.

In one embodiment the cleavage of the single stranded detector DNA is detected visually on a lateral flow system.

Lateral flow systems are typically based on flow strips. A flow strip may comprise one or more pads for holding a sample fluid, and may further comprise one or more detector reagents for detecting a target moiety in the sample fluid. The pads may be based on a series of capillary beds, such as pieces of porous paper, microstructured polymer, or sintered polymer. Each of these pads may have the capacity to transport sample fluid spontaneously, e.g., via capillary action. A sample to be analysed may be added to the proximal end of the strip. Liquid-phase elements of the sample (which may be dissolved, suspended, emulsified or in any other liquidized format) may migrate to a region of the pad wherein a detector reagent has been immobilized, typically consisting of a protein linked passively or covalently to a signal molecule or particle, typically a colloidal gold, or a colored, fluorescent or paramagnetic monodisperse latex particle. The signal reagent can also be another reagent, including non-particulates (e.g., soluble, directly labeled fluorophores gels). This label may be conjugated to one of the specific biological components of the assay, either an antigen or an antibody, depending on the assay format of the specific flow strip. The liquid phase sample re-mobilizes the dried conjugate material causing it to incorporate into the liquid phase sample material, and an analyte in the sample interacts with the conjugate. The conjugate material may be proteins, e.g., antibody or antigen, which have been laid down in bands or stripes in specific areas of a pad where they serve to capture the components of the liquid phase sample, the analyte and conjugate, as they migrate past, through or over the capture lines. Excess liquid phase materials (sample and reagents) continue to migrate across the strip, past the capture lines and may be entrapped in a pad near the other end of the flow strip. Test results may be developed on the reaction matrix and may be represented as the presence or absence of test indicia (typically continuous lines) of captured conjugate which are read either by eye or using a reader device. Some of the conjugated particles may not be captured at a capture line, and will continue to flow toward a second line of immobilized detector reagents, the control line. This control line typically comprises another detector reagent that is specific for the conjugate antibody on the conjugate. Binding of the conjugated particles to this detector reagent generates a control signal (typically a continuous line).

In one embodiment the absence of a test line on a lateral flow system indicates that a polynucleotide analyte is present in a sample.

In some embodiments, steps (a) and (b) of the methods herein are performed in separate reaction vessels, and the products of those reaction vessels are mixed before proceeding with step (c).

In some embodiments, steps (a) and (b) of the methods herein are performed in a single reaction vessel. Steps (a) and (b) may proceed concurrently in the single reaction vessel. Alternatively, the reagents required for step (b) (i.e., the nucleic acid ligase and nucleic acid adaptor) may be added to the reaction vessel after step (a) has proceeded for a known duration. In some embodiments step (c) is also performed in the same reaction vessel as steps (a) and (b). Steps (a), (b) and (c) may proceed concurrently in the single reaction vessel. Alternatively, the Cas nuclease, guide RNA and single stranded detector DNA may be added to the reaction vessel after step (b) has proceeded for a known duration.

In some embodiments the method herein further comprises amplifying the polynucleotide analyte before step (a).

Various amplification methods and components will be known to one of ordinary skill in the art and any convenient method can be used (see, e.g., Zanoli and Spoto, Biosensors (Basel). 2013 March; 3 (1): 18-43; Gill and Ghaemi, Nucleosides, Nucleotides, and Nucleic Acids, 2008, 27:224-243; Craw and Balachandrana, Lab Chip, 2012, 12, 2469-2486). Nucleic acid amplification can comprise polymerase chain reaction (PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR, intersequence-specific PCR (ISS-PCR), whole genome amplification (WGA), inverse PCR, and thermal asymmetric interlaced PCR (TAIL-PCR).

In some embodiments the amplification is isothermal amplification. The term “isothermal amplification” indicates a method of nucleic acid (e.g., DNA) amplification (e.g., using enzymatic chain reaction) that uses a single temperature incubation thereby obviating the need for a thermal cycler. Isothermal amplification is a form of nucleic acid amplification which does not rely on the thermal denaturation of the target nucleic acid during the amplification reaction and hence may not require multiple rapid changes in temperature. Isothermal nucleic acid amplification methods can therefore be carried out inside or outside of a laboratory environment. By combining with a reverse transcription step, these amplification methods can be used to isothermally amplify RNA.

Examples of isothermal amplification methods include but are not limited to: loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA).

In some cases, the amplification is recombinase polymerase amplification (RPA). Recombinase polymerase amplification (RPA) uses two opposing primers (much like PCR) and employs three enzymes—a recombinase, a single-stranded DNA-binding protein (SSB) and a strand-displacing polymerase. The recombinase pairs oligonucleotide primers with homologous sequence in duplex DNA, SSB binds to displaced strands of DNA to prevent the primers from being displaced, and the strand displacing polymerase begins DNA synthesis where the primer has bound to the target DNA. Adding a reverse transcriptase enzyme to an RPA reaction can facilitate detection RNA as well as DNA, without the need for a separate step to produce cDNA.

In a transcription mediated amplification (TMA), an RNA polymerase is used to make RNA from a promoter engineered in the primer region, and then a reverse transcriptase synthesizes cDNA from the primer. A third enzyme, e.g., Rnase H can then be used to degrade the RNA target from cDNA without the heat-denatured step. This amplification technique is similar to Self-Sustained Sequence Replication (3SR) and Nucleic Acid Sequence Based Amplification (NASBA), but varies in the enzymes employed.

In one embodiment, a polynucleotide is amplified isothermally using loop mediated amplification (LAMP). LAMP employs a thermostable polymerase with strand displacement capabilities and a set of four or more specific designed primers. Each primer is designed to have hairpin ends that, once displaced, snap into a hairpin to facilitate self-priming and further polymerase extension. In a LAMP reaction, though the reaction proceeds under isothermal conditions, an initial heat denaturation step is required for double-stranded targets. In addition, amplification yields a ladder pattern of various length products. For yet another example, a strand displacement amplification (SDA) combines the ability of a restriction endonuclease to nick the unmodified strand of its target DNA and an exonuclease-deficient DNA polymerase to extend the 3′ end at the nick and displace the downstream DNA strand.

In one embodiment, a polynucleotide is amplified isothermally using helicase-dependent amplification (HDA). HDA utilizes a thermostable helicase (Tte-UvrD) rather than heat to unwind dsDNA to create single-strands that are then available for hybridization and extension of primers by polymerase.

Disclosed herein is a method of detecting a polynucleotide analyte in a sample, the method comprising
a) i) contacting the sample comprising the polynucleotide analyte with a first nucleic acid probe and a second nucleic acid probe that are configured to form a cleavage structure in the presence of the polynucleotide analyte; and
   ii) a structure-specific nucleic acid cleaving agent;
wherein formation of the cleavage structure allows cleavage of the first nucleic acid probe by the cleaving agent to release a 5′ flap from the first nucleic acid probe;
b) ligating the 5′ flap to a nucleic acid adaptor to form an adaptor-ligated product, wherein the nucleic acid adaptor comprises a double-stranded region and a 3′overhang extension that is complementary to the 5′ flap and wherein the 3′ overhang extension hybridizes to the 5′ flap to the nucleic acid adaptor;
c) contacting the adaptor-ligated product with:
   i) a type V CRISPR/Cas effector protein;
   ii) a guide RNA comprising a region that binds to the type V CRISPR/Cas effector protein and a guide sequence that is complementary to a portion of the 5′ flap and a portion of the nucleic acid adaptor that is ligated to and adjacent to the 5′ flap; and
   iii) a single stranded detector DNA; and d) measuring a detectable signal produced by cleavage of the single stranded detector DNA by the type V CRISPR/Cas effector protein to detect the adaptor-ligated product, thereby detecting the polynucleotide analyte in the sample.

The method for detection of DNA/RNA has various diagnostic applications as it can be used to identify any genetic sequence, without using a sequencer. Detection and sub-typing of bacterial and viruses pathogens in various human, food and environmental samples is possible with this method. The detection method is also able to distinguish between different variants of such pathogens, such as antibiotic-resistant or vaccine-resistant variants. Moreover, detection of genetic mutations in the human genome linked to the development of diseases as well as cell-free DNA/RNA in different samples is also feasible with this method.

The method of the present invention may be useful for detecting the presence or absence of one or more polynucleotide analytes in one or more samples known to contain or suspected of containing the polynucleotide analytes. The method can also be used to quantify the amount of polynucleotide analytes within the sample. The method is useful for detecting polynucleotide target in a sample such as for example RNA, MRNA, rRNA, plasmid DNA, viral DNA, bacterial DNA, and chromosomal DNA.

The term “polynucleotide analyte” may be any polynucleotide that may be detected or analyzed by a method as defined herein. The analyte may be naturally-occurring or synthetic. A polynucleotide analyte may be present in a sample obtained using any methods known in the art. In some cases, a sample may be processed before analyzing it for a polynucleotide analyte. The polynucleotide may include DNA, RNA, peptide nucleic acids, and any hybrid thereof, where the polynucleotide contains any combination of deoxyribo- and/or ribo-nucleotides. Polynucleotides may be single stranded or double stranded, or contain portions of both double stranded or single stranded sequence. Polynucleotides may contain any combination of nucleotides or bases, including, for example, uracil, adenine, thymine, cytosine, guanine, inosine, xanthine, hypoxanthine, isocytosine, isoguanine and any nucleotide derivative thereof. As used herein, the term “nucleotide” may include nucleotides and nucleosides, as well as nucleoside and nucleotide analogs, and modified nucleotides, including both synthetic and naturally occurring species. Polynucleotides may be any suitable polynucleotide, including but not limited to cDNA, mitochondrial DNA (mtDNA), messenger RNA (mRNA), ribosomal RNA (rRNA), transfer RNA (tRNA), nuclear RNA (nRNA), small interfering RNA (siRNA), small nuclear RNA (snRNA), small nucleolar RNA (snoRNA), small Cajal body-specific RNA (scaRNA), microRNA (miRNA), double stranded (dsRNA), ribozyme, riboswitch or viral RNA. Polynucleotides may be contained within any suitable vector, such as a plasmid, cosmid, fragment, chromosome, or genome. The polynucleotide analyte can be a nucleic acid endogenous to the cell. As another example, the polynucleotide analyte can be a nucleic acid introduced to or expressed in the cell by infection of the cell with a pathogen, for example, a viral or bacterial genomic RNA or DNA, a plasmid, a viral or bacterial mRNA, or the like.

In some embodiments the polynucleotide analyte is a DNA or an RNA. In some embodiments the polynucleotide analyte is single stranded or double stranded.

In one embodiment the polynucleotide analyte comprises a single nucleotide polymorphism (SNP).

In one embodiment the polynucleotide analyte is a viral nucleic acid. In one embodiment, the polynucleotide analyte is a viral nucleic acid from SARS-CoV-2. The SARS-CoV-2 genome consists of ˜30 kb positive single-stranded RNA with a 5 ‘-cap structure and 3’ poly-A tail containing several genes characteristic of coronaviruses, such as S (spike), E (envelope), M (membrane), and N (nucleocapsid) genes. Other elements of the genome, such as ORF1a and ORF1b, encode non-structural proteins, including RNA-dependent RNA polymerase (RdRp).

As used herein, the term “sample” includes tissues, cells, body fluids and isolates thereof etc., isolated from a subject, as well as tissues, cells and fluids etc. present within a subject (i.e. the sample is in vivo). Examples of samples include: whole blood, blood fluids (e.g. serum and plasm), lymph and cystic fluids, sputum, stool (or fecal), tears, mucus, hair, skin, ascitic fluid, cystic fluid, urine, nipple exudates, nipple aspirates, sections of tissues such as biopsy and autopsy samples, frozen sections taken for histologic purposes, archival samples, explants and primary and/or transformed cell cultures derived from patient tissues etc.

The terms “detecting”, “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. The method as defined herein may comprise measuring or visualising the levels of two or more polynucleotide analytes in a sample.

The methods may be used for any purpose for which detection of viral, bacterial or other nucleic acids is desirable, including diagnostic and prognostic applications, such as in laboratory and clinical settings. In some embodiments, the methods may be used for detection of a nucleic acid for genotyping.

In some embodiments, the nucleic acid to be detected is diagnostic for a disease state. The disease state can be an infection, an organ disease, a blood disease, an immune system disease, a cancer, a brain and nervous system disease, an endocrine disease, a pregnancy or childbirth-related disease, an inherited disease, or an environmentally-acquired disease, cancer, or a fungal infection, a bacterial infection, a parasite infection, or a viral infection. Thus, in some embodiments, the method is useful for detecting a nucleic acid (e.g., DNA or RNA) from a bacterium, fungus, virus (e.g., caused by a double-stranded RNA virus, a positive sense RNA virus, a negative sense RNA virus, a retrovirus, etc.), or parasite.

Exemplary viruses that can be detected include, without limitation, Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterpro virus, Rhizidovirus, a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In some embodiments, the virus is coronavirus (e.g., SARS-Cov-2), SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In some embodiments, the nucleic acid to be detected can be associated with a pathogen, including pathogenic bacteria such as, E. faecalis, E. faecium, Listeria monocytogenes, Campylobacter jejuni, Staphylococcus aureus (e.g., MRSA), E. coli O157:H7, Borrelia burgdorferi, Helicobacter pylori, Ehrlichia chaffeensis, Clostridium difficile, Vibrio cholerae 0139, Salmonella enterica, Bartonella henselae, Streptococcus pyogenes, Chlamydia pneumoniae, Clostridium botulinum, Corynebacterium amycolatum, Klebsiella pneumonia, Vibrio vulnificus, and Parachlamydia.

Provided herein are also methods of treating the disease following detection of the disease.

By “subject” or “patient” is meant any single subject for which therapy is desired, including humans, cattle, horses, pigs, goats, sheep, dogs, cats, guinea pigs, rabbits, chickens, insects and so on. Also intended to be included as a subject are any subjects involved in clinical research trials not showing any clinical sign of disease, or subjects involved in epidemiological studies, or subjects used as controls.

Disclosed herein is a kit for performing a method as disclosed herein. The kit may comprise i) a first nucleic acid probe comprising a 3′ portion complementary to a first portion of the polynucleotide analyte and a 5′ portion that is not complementary to and does not hybridize to the polynucleotide analyte; ii) a second nucleic acid probe comprising a 5′ portion complementary to a second portion of said polynucleotide analyte and a 3′ portion that is not complementary to and does not hybridize to the polynucleotide analyte, wherein said first portion of the polynucleotide analyte is 5′ to and contiguous with the second portion of the polynucleotide analyte; and iii) a structure-specific nucleic acid cleaving agent. The structure-specific nucleic acid cleaving agent may be a polypeptide comprising an amino acid sequence having at least 80% sequence identity to an amino acid sequence set forth in SEQ ID NO: 1. The kit may further comprise a nucleic acid adaptor for forming an adaptor-ligated product, wherein the nucleic acid adaptor comprises a double-stranded region and a 3′overhang extension that is complementary to the 5′ flap and wherein the 3′ overhang extension is capable of hybridizing to the 5′ flap to the nucleic acid adaptor. The kit may further comprise i) a type V CRISPR/Cas effector protein: ii) a guide RNA comprising a region that binds to the type V CRISPR/Cas effector protein and a guide sequence that is complementary to a portion of the 5′ flap and a portion of the nucleic acid adaptor that is ligated to and adjacent to the 5′ flap; and iii) a single stranded detector DNA. A detectable signal produced by cleavage of the single stranded detector DNA by the type V CRISPR/Cas effector protein can be measured by a detector. The kit may further comprise one or more buffer components, metabolites, and/or other reaction components necessary to perform the methods defined herein. The kit may further comprise a lateral flow device or system for detecting the signal generated by cleavage of the single stranded detector DNA. The kit may further comprise instructions for performing the methods defined herein.

Disclosed herein is a kit for detecting a polynucleotide analyte in a sample, comprising a structure-specific nucleic acid cleaving agent, a nucleic acid ligase and a type V CRISPR/Cas effector protein. The structure-specific nucleic acid cleaving agent may be a polypeptide comprising an amino acid sequence having at least 80% sequence identity to an amino acid sequence set forth in SEQ ID NO: 1. The kit may further comprise a first nucleic acid probe and a second nucleic acid probe, wherein the first and second nucleic acid probes are configured to form a cleavage structure in the presence of the polynucleotide analyte. The kit may further comprise a first adaptor polynucleotide and a second adaptor polynucleotide, wherein the first and second adaptor polynucleotides are configured to form a nucleic acid adaptor on hybridization. The kit may further comprise a guide RNA configured to bind to the type V CRISPR/Cas effector protein and a product of ligation of a nucleic acid adaptor and a cleavage product of the structure-specific nucleic acid cleaving agent. The kit may further comprise a single stranded detector DNA. The kit may further comprise one or more buffer components, metabolites, and/or other reaction components necessary to perform the methods defined herein. The kit may further comprise a lateral flow device or system for detecting the signal generated by cleavage of the single stranded detector DNA. The kit may further comprise instructions for performing the methods defined herein.

In some embodiments, a method or kit herein exhibits an attomolar (aM) sensitivity of detection. In some cases, a subject composition or method exhibits a femtomolar (fM) sensitivity of detection. In some cases, a subject composition or method exhibits a picomolar (pM) sensitivity of detection. In some cases, a subject composition or method exhibits a nanomolar (nM) sensitivity of detection.

In some embodiments, the threshold of detection for a target polynucleotide, using a method defined herein, is 1 nM or less. The term “threshold of detection” is used herein to describe the minimal amount of target polynucleotide that must be present in a sample in order for detection to occur. Thus, as an illustrative example, when a threshold of detection is 1 nM, then a signal can be detected when a target DNA is present in the sample at a concentration of 1 nM or more. In some embodiments, a method of the present disclosure has a threshold of detection of 500 pM or less, 100 pM or less, 50 pM or less, 10 pM or less, 5 pM or less, 1 pM or less, 500 fM or less, 100 fM or less, 50 fM or less, 10 fM or less, 5 fM or less, 1 fM or less, 500 aM or less, 100 aM or less, 50 aM or less, 10 aM or less, or 1 aM or less.

In some embodiments, the threshold of detection is between about 100 pM to about 1 nM, between about 10 pM to about 100 pM, between about 1 pM to about 10 pM, between about 100 fM to about 1 pM, between about 10 fM to about 100 fM, between about 1 fM to about 10 fM, between about 100 aM to about 1 fM, between about 10 aM to about 100 aM, or between about 1 aM to about 10 aM.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Those skilled in the art will appreciate that the invention described herein is susceptible to variations and modifications other than those specifically described. It is to be understood that the invention includes all such variations and modifications, which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### Examples

### Methods

### Bacterial Strains, Plasmids and Oligonucleotides Used in the Study

E. coli TOP10 was used as the cloning host and E. coli BL21(DE3) was used for protein expression. Both bacterial strains were grown in Luria-Bertani (LB) broth at 37° C. with shaking at 220 rpm unless stated otherwise.

pET28b was used as the cloning vector to express the proteins in E. coli BL21(DE3). Cas12 was obtained from pMBP-LbCas12a, which was a gift from Jennifer Doudna (Addgene plasmid #113431). The Tth pol (GenBank accession no. WP_011228405), hFEN1 (GenBank accession no. NP_004102), MjaFEN (GenBank accession no. WP_010870964) and T5 exo (GenBank accession no. YP_006958) sequences were codon optimized for expression in E. coli and synthesized as gblocks from Integrated DNA Technologies (IDT).

The sequence of the oligos and primers for LAMP, HDA and FEN (flap and invading primers) are provided in Tables 1-4.

### Protein Purification

All proteins were purified by using the His tag attached at their C-termini. For Cas12 purification, E. coli BL21 (DE3) (pET28b-Cas12) was grown overnight in Luria-Bertani (LB) broth supplemented with kanamycin (50 μg/mL). After overnight growth, the culture was diluted 10-fold in Terrific Broth supplemented with kanamycin and grown at 37° C. until the OD600 reached ˜0.3-0.4. Following this, 100 mM IPTG was added to the cultures and grown further for 24 h at 23° C. The cells were collected by centrifugation and resuspended in lysis buffer (50 mM Tris-Cl (pH 8), 300 mM NaCl, EDTA-free protease inhibitor tablets). The cells were lysed using a homogenizer by passing the cells at 23,000 bar 5-10 times. The lysate was centrifuged, and the supernatant was subjected to protein purification using a HisTrap HP column connected to an FPLC instrument. Elution was performed with lysis buffer supplemented with 1 M imidazole, following a gradient run over 2 h. Different fractions were collected and run on an SDS-PAGE gel to confirm protein purification. The purified protein was washed with lysis buffer without protease inhibitor tablets to remove imidazole and then concentrated and flash frozen for storage.

FEN enzyme purification was performed in the same manner with slight modifications. The growth medium for protein expression was changed to LB, and the protein was expressed at 37° C. for 4-5 h. Cell lysis was performed using Y-PER (Thermo Fisher), and the soluble proteins were purified using Ni-NTA resin in a PD-10 column. Elution was performed using lysis buffer supplemented with 50 mM, 100 mM, 200 mM, and 500 mM imidazole. Purified proteins (final concentration 1 μM) were stored in a buffer comprising 50% glycerol, 0.1 mM EDTA, 1 mM DTT, and 0.1% Triton-X-100 at −20° C.

### FEN Activity Assay

The activity of various FEN enzymes was evaluated in an isothermal reaction comprising reaction buffer RB (50 mM Tris-Cl (pH 8), 100 mM NaCl, 10 mM MgCl₂), 100 nM flap primer, 20 nM invading primer, 0.2% Triton-X-100, 50 nM purified FEN enzyme (unless otherwise stated), and 1 nM target template (unless otherwise stated) in a 20 μL reaction incubated at 65° C. for the appropriate time. When using the flap primer with a conjugated fluorophore and quencher, the fluorescence (ex: 485 nm, em: 535 nm) was measured every 5 min.

### Ligation Assay

To ligate the synthesized flap oligo to the dsDNA adaptor, the latter was formed by annealing the 5′ p adaptor and ds convertor (final concentration 200 nM) in 1×RB. For this, the reaction mixture was heated to 98° C. and cooled to room temperature at 0.1° C./s in a thermocycler. The ligation was carried out by adding 1×RB, 5 nM dsDNA adaptor, 5 nM flap oligo, and the appropriate additive and co-factor depending upon the ligase used. For T4 DNA ligase and T4 RNA ligase (NEB), 1 mM DTT and 1 mM ATP (NEB) were added to the reaction. For Taq DNA ligase (NEB), 1 mM NAD (Sigma Aldrich) and 10 mM DTT were added to the reaction. The amounts of ligase used were 5 U of T4 DNA ligase, 10 U of T4 RNA ligase, and 80 U of Taq DNA ligase. The incubation temperatures were 22° C. for T4 DNA ligase, 37° C. for T4 RNA ligase, and 65° C. for Taq ligase.

### Cas12-sgRNA Reaction

The Cas12-sgRNA complex was formed by incubating 3.75 μL of purified Cas12 (100 μM) and 0.3125 μL of 1 mM sgRNA in 2 μL of 1×RB with 20 mM DTT (RB-DTT) made up to a final volume of 20 μL with nuclease-free water. The incubation was performed at 37° C. for 30 min. Following this, the Cas12-sgRNA complex was diluted to 375 μL with water to achieve a final Cas12: sgRNA concentration ratio of 1 μM: 0.833 μM.

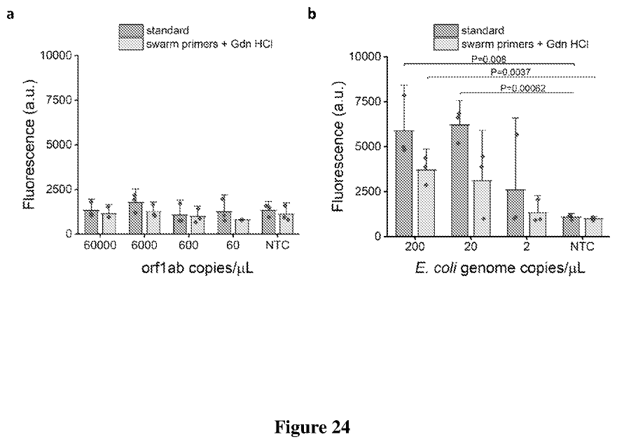

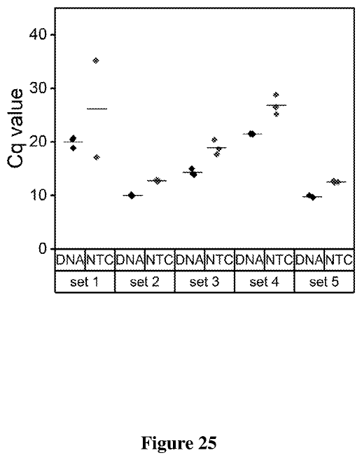

For the reaction with the Cas12-sgRNA complex, 3 μL of the complex formed as above, 4 μL of the fluorophore-quencher reporter (1 μM), and 0.7 μL of 1×RB were added to the reaction and incubated at 37° C., with fluorescent measurements taken every 3 min (ex: 485 nm, em: 535 nm). For the data in FIGS. 21, 23 and 24, 1.5 μL of Cas12-sgRNA complex and 2 μL of the fluorophore-quencher reporter were used.

### LAMP Assay

To perform LAMP, a Warmstart LAMP kit (DNA and RNA) from NEB was used, and the assay was performed according to the manufacturer's instructions. LAMP primers were designed using either PrimerExplorer v5 or the NEB LAMP primer design tool. To biotinylate the LAMP product for subsequent purification with streptavidin agarose beads, the primers FIP, BIP, LF, and LB were biotinylated at the 5′-end. After LAMP, 10 μL of DNA product was purified by adding an equal volume of Pierce Streptavidin Agarose resin resuspended in B&W buffer (10 mM Tris-Cl (pH 7.5), 1 mM EDTA and 2 M NaCl). The sample was diluted further by adding 30 μL of 0.5×B&W buffer and incubating the mixture for 30 min at room temperature for biotin-streptavidin conjugation. After 30 min, the supernatant was removed after the beads settled. These beads were subsequently used for FELICX.

### HDA Assay

HDA was performed using the IsoAmp II Universal tHDA kit from NEB. The assay was performed according to the manufacturer's instructions, with primers designed using the PrimerQuest tool from IDT. For all HDA reactions, MgSO₄ (4 mM), NaCl (40 mM) and the appropriate primers (200 nM) were used. After assay completion, the DNA product was used directly for FELICX or purified using the ChargeSwitch PCR Clean-Up kit from Invitrogen followed by FELICX. When RNA was used as the template, 0.25 μL Warmstart RTx (NEB) was added to every 10 μL of HDA assay mix.

### FELICX (Flap Endonuclease, Taq LIgase and CRISPR-Cas for Diagnostics (X))

In the first step of FELICX, amplified DNA was added to the FEN+Taq mix comprising of: 2 μL of flap primer (1 μM), 0.4 μL of invading primer (1 μM), 0.5 μL of dsDNA adaptor (200 nM, as described in the ligation assay), 2 μL of RB-DTT buffer, 1 μL of Triton-X-100 (2%), 1 μL of NAD (20 mM), 0.5-1 μL of FEN, and 0.5 μL of Taq ligase, made up to a final volume of 20 μL with nuclease-free water. The reaction was carried out at 65° C. for the appropriate duration. Following this, the reaction mixture was added to the Cas12-sgRNA mix, comprising 3 μL of Cas12-sgRNA complex (as described for the Cas12-sgRNA reaction), 4 μL of FAM-IBFQ reporter probe (1 μM), and 0.7 μL of RB-DTT and incubated at 37° C. with the fluorescence (ex: 485 nm, em: 535 nm) measured in a microplate reader every 3 min.

For the lateral flow strips, the FAM-IBQF reporter was replaced with 0.25 μL of FAM-Bio reporter (1 μM) or 0.1 μL of DIG-Bio reporter (1 μM). Following Cas12 cleavage, the reaction mix was run on PCRD Flex strips from Abingdon Health according to the manufacturer's instructions. Band intensity was quantified using ImageJ.

### SNP Detection

To detect the T478K mutation in the receptor-binding domain (RBD) of SARS-CoV-2, the mutation was introduced in pCDNA3-SRARS-CoV-2-S-RBD-Fc, which carries the RBD region (˜1.5 KB) of the virus, by overlap PCR using the primers spike-T478K-F/R. After confirming mutagenesis by sequencing, the wild-type and mutated RBD regions were PCR amplified using the primers spike-F/R and an equal amount of plasmid. Following amplification, 50-150 ng of purified PCR product was used as the template for FELICX, which was performed as previously described with slight modifications. The volume of the flap primers (WT and mut probes) was increased to 3 μL (final concentration 150 nM). Both templates were analyzed with the WT and mut probes. In the case of the lateral flow strips, the FAM-Bio reporter was used in the Cas12-sgRNA cleavage reaction for the WT probe samples and the DIG-Bio reporter was used for the mut probe samples.

### Detection of Genes in Mammalian Cells

The C666-1 cell line was kindly provided by Dr Joshua Tay from the Department of Otolaryngology, Yong Loo Lin School of Medicine, National University of Singapore, and the HK-1 cell line was a gift from A/Prof Zhong Yong Liang from the Department of Microbiology & Immunology, Yong Loo Lin School of Medicine, National University of Singapore. Both cell lines were grown in Gibco BenchStable RPMI 1640 (Life Technologies, Catalog #A4192301) supplemented with 10% FBS (Biowest, Catalog #S181H) and 1% penicillin-streptomycin (Gibco, Catalog #15140122) at 37° C. in 5% CO₂. After reaching 70-80% confluency, the cells were trypsinized and washed with RPMI, and cell numbers were counted using a Luna cell counter. A fixed number of C666-1 or HK-1 cells were lysed by incubation in lysis buffer (10 mM Tris-Cl (pH 7.5), 200 μg/mL proteinase K, 0.1% SDS, 2 mM CaCl₂)) at 65° C. for 10 min. After lysis, the genetic material was purified using a ChargeSwitch PCR Clean-Up kit and subjected to FELICX as described above. The fluorescence measured after 30 min of the Cas12-sgRNA reaction was then reported.

Detection of Genes in K. pneumonia

An overnight culture of K. pneumoniae DSM 2026 grown in LB broth at 37° C. in shaking conditions was diluted to obtain the required c.f.u. based on the formula OD600=˜108 c.f.u. HK-1 cells were grown in Gibco BenchStable RPMI 1640 (Life Technologies, Catalog #A4192301) supplemented with 10% FBS (Biowest, Catalog #S181H) and 1% penicillin-streptomycin (Gibco, Catalog #15140122) at 37° C. in 5% CO2. After reaching 70-80% confluency, the cells were trypsinized and washed with RPMI, and cell numbers were counted using a Luna cell counter. Simulated clinical samples were prepared by mixing K. pneumoniae with HK-1 cells, as needed, and lysing the samples by incubation in lysis buffer (10 mM Tris-Cl (pH 7.5), 200 μg/mL proteinase K, 0.1% SDS, 2 mM CaCl2) at 65° C. for 10 min. After lysis, the genetic material was purified using a ChargeSwitch PCR Clean-Up kit and subjected to FELICX as previously described. The fluorescence after 30 min of the Cas12-sgRNA reaction was then reported.

### Detection of Cancer Biomarkers

To prepare the template, overlapping primers (scgb1-F, scgb2-R, scgb3-F and scgb4-R) were designed and PCR amplified with scgb2a2-T7-F and scgb2a2-R. The final construct incorporates a T7 promoter at the 5′-end of SCGB2A2, enabling in vitro transcription by the Hiscribe T7 High Yield RNA Synthesis kit (NEB). The transcribed product was purified by a Monarch RNA Clean-Up kit (NEB) and used for FELICX. To simulate clinical samples, RNA was quantified and spiked into Fetal Bovine Serum (FBS) South America, Heat Inactivated (Biowest, Catalog #S181H). The simulated samples were processed with a ChargeSwitch PCR Clean-Up kit to purify the spiked RNA followed by HDA with RTx and FELICX, as previously described. The fluorescence after 30 min of the Cas12-sgRNA reaction was then reported.

### Example 1: Characterization and Optimization of FEN

To develop FELICX, the cleavage activity of FEN was first characterized and optimized. FENs are a class of enzymes ubiquitous in both prokaryotes and eukaryotes. The main role of FEN is in DNA replication, where it removes the RNA primer from the 5′-end of the Okazaki fragments that are subsequently joined together to form the lagging strand. In bacteria, DNA polymerase I—which consists of an N-terminal 5′ nuclease and a C-terminal polymerase—plays the role of FEN via its N-terminal domain. In archaea and mammalian cells, there is a dedicated FEN enzyme similar to the N-terminal domain of bacterial DNA polymerase I.

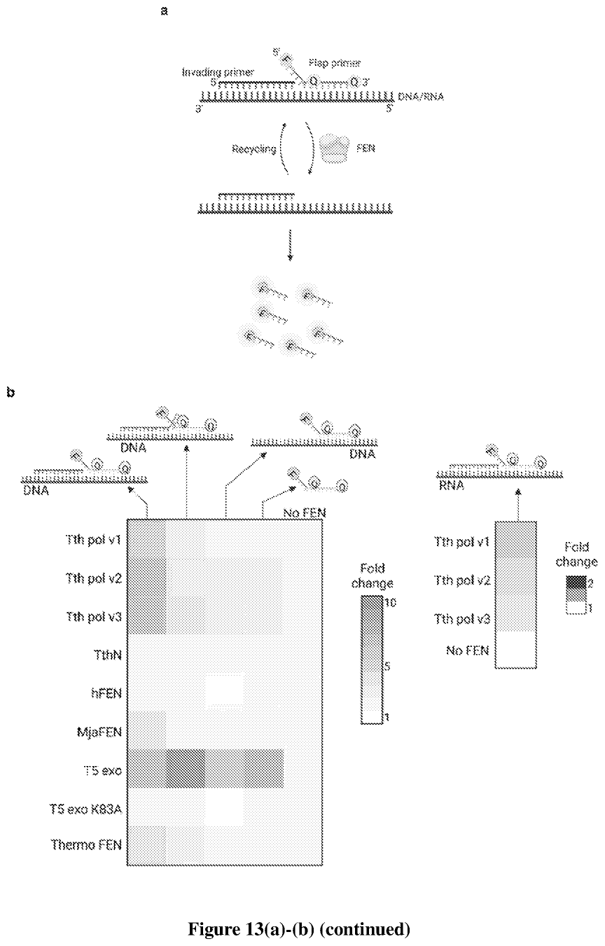

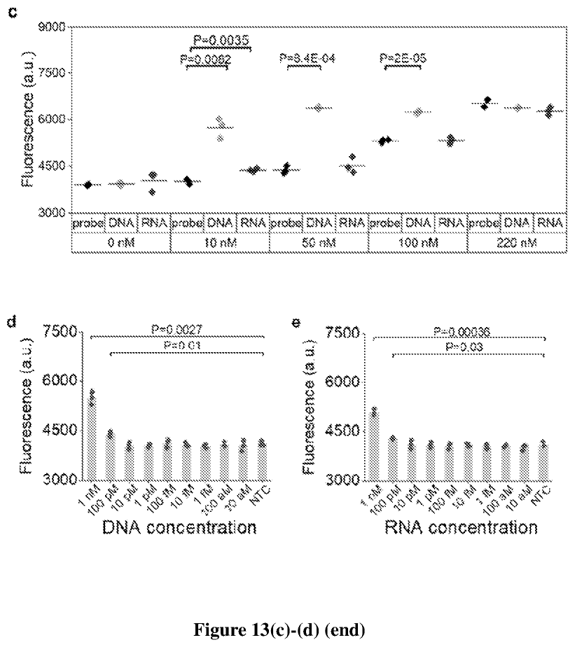

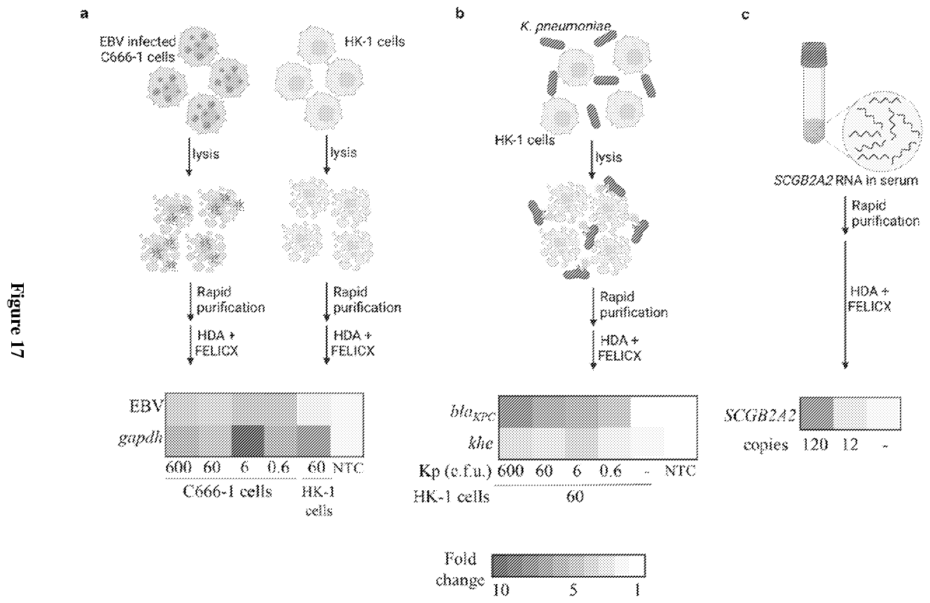

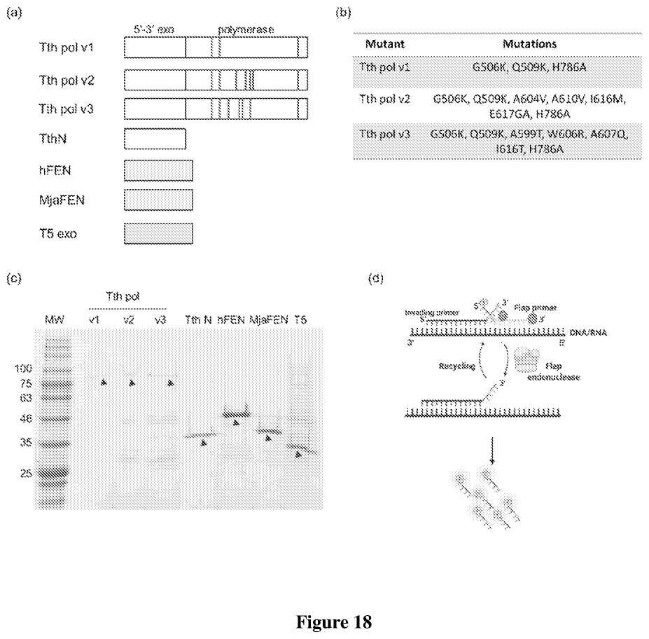

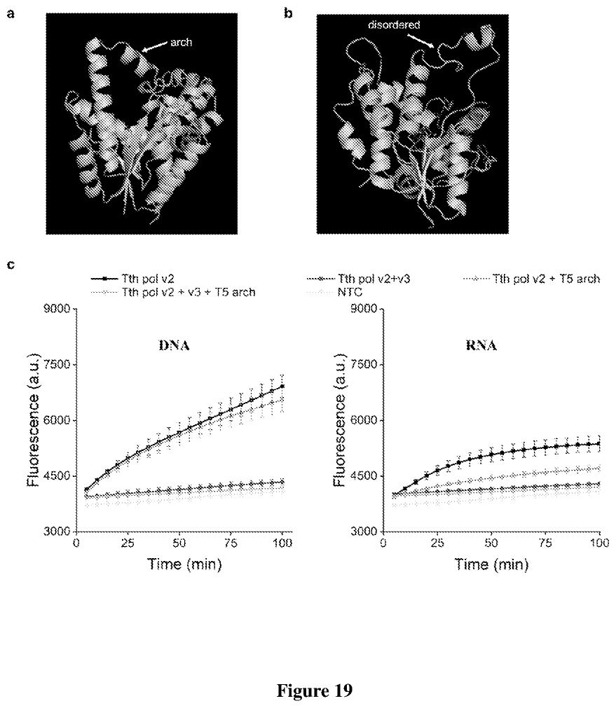

FEN has been used to detect both DNA and RNA, although it has several limitations, such as its slow speed, need for initial denaturation to assemble the oligos on the target, and reliance on a fluorescence reader for signal detection. DNA polymerase I from Thermus thermophilus (Tth pol) is a well-characterized FEN that can recognize the flap structure on both DNA and RNA targets. To determine if Tth pol is the best candidate for FELICX, a panel of Tth pol variants was expressed and purified: the N-terminal domain of Tth pol (TthN), human FEN1 (hFEN), archaeal Methanococcus jannaschii FEN1 (MjaFEN), bacteriophage T5 exonuclease (T5 exo) (FIGS. 18a and c) and compared their activities with a commercially available thermostable FEN (thermo FEN) (FIG. 13). Previously, wild-type Tth pol was mutated (G506K, Q509K, H786A) to enable RNA recognition and inactivate the polymerase domain. This mutant is named Tth pol v1 in this study. Mutations in Taq polymerase have also been reported to expand its substrate spectrum, although their effect on the FEN activity of the enzyme was not studied. These mutations lie in the polymerase domain of the enzyme and may affect its substrate binding and, consequently, FEN activity. Due to the high similarity between the Taq and Tth polymerases, corresponding mutations in Tth pol might have a similar effect on the enzyme activity. Therefore, Tth pol v1 was further mutated to include the A604V, A610V, I616M, and E617GA mutations, creating Tth pol v2. Similarly, Tth pol v1 was mutated to include the A599T, W606R, A607Q, and 1616T mutations to create Tth pol v3 (FIG. 18b). TthN was also created, comprising only of the N-terminal domain (amino acids 1-307) of full-length Tth pol, and tested its FEN activity based on a previous report showing that the N-terminal domain of Taq polymerase alone exhibits FEN activity.

To quantify FEN activity, a flap primer with a 5′ fluorophore and two attached quenchers—3′ and internal—was used. This primer would generate a fluorescence signal upon cleavage by FEN (FIG. 13a). For all the FEN enzymes, the enzyme activity on different configurations of the flap and invading primers was tested: the 5′-flap of the flap primer and an invading primer (FIG. 13a), the double flap wherein both the flap and invading primers have flaps (FIG. 18d), as well as in the absence of the target-specific invading primer. The nonspecific activity of FEN on the fluorophore-quencher flap primer alone was also measured. For the target, a partial orflab DNA of SARS-CoV-2 with low similarity to the SARS viral genome was used. The reaction was performed at 65° C., which is the melting temperature of the flap primer used in this study.

As seen in the left panel of FIG. 13b, the activity of Tth pol v1, v2, and v3 was greatly reduced when the double flap substrate was used (FIG. 18d) and when the invading primer was missing, suggesting that the configuration shown in FIG. 2a is the best for these enzymes. Among these variants of Tth pol, Tth pol v2 showed the highest activity, followed by Tth pol v3 and v1 (FIG. 13b), on the DNA substrate to which the flap primer and an invading primer without the flap were bound, validating our hypothesis that mutations introduced in the enzyme affect the enzyme activity. Fluorescence signals were also observed in the case of the flap primer alone (FIG. 13b), indicating that the enzymes had low nonspecific activity along with high specific FEN activity. Surprisingly, TthN did not show any activity for any of the substrates tested, contrary to a previous study performed using the highly similar N-terminal domain of Taq polymerase. hFEN did not show any activity, likely due to the high reaction temperature, while the thermophilic MjaFEN displayed poor activity for the substrate with the 5′-flap of the flap primer and an invading primer (FIG. 13b). For T5 exo, strong fluorescence signals were observed for all substrates, including the flap primer alone, almost immediately after initiating the reaction. This indicates that the enzyme has very high exonuclease activity that masks its FEN activity. To abolish its exonuclease activity, the K83 residue, which has been shown to be essential for exonuclease but not endonuclease activity, was mutated. However, in this study, the K83A mutation completely inactivated the enzyme, abolishing both endo- and exonuclease activities (FIG. 13b). Meanwhile, thermos FEN showed a similar trend of activity with the various DNA substrates as Tth pol. However, compared to Tth pol and its variants, it had lower activity. These results indicate that Tth pol v2 is the best candidate with high specific FEN activity and that the ideal substrate for the enzyme is the DNA target bound to a flap primer and an invading primer without a flap.

Next, the best FEN candidate, Tth pol v2, was selected and its activity was compared with the other Tth pol variants, v1 and v3, on an RNA substrate to which the flap primer and the invading primer with the flap were bound. Similar to the previous experiment, the partial orflab RNA of SARS-CoV-2 was used for the assay. As seen in the right panel of FIG. 13b, Tth pol v1 and v2 performed better than Tth pol v3, although weaker signals were observed for all three enzymes compared to the DNA substrate. Based on the FEN activity observed on both DNA and RNA substrates, Tth pol v2 was selected as the FEN for FELICX. Further mutagenesis of Tth pol v2 did not significantly improve the FEN activity of the enzyme.

We further characterized Tth pol v2 by evaluating the effect of enzyme concentration on FEN activity on both DNA and RNA substrates and the nonspecific activity on the flap primer alone (labeled as probe). We observed that with increasing enzyme concentrations, there was a concomitant increase in nonspecific activity, as indicated by the increasing fluorescence with the probe alone (FIG. 13c). At 10 nM, non-specific activity was minimal, with high FEN activity observed for both DNA and RNA substrates. Beyond this concentration, non-specific activity significantly increased, subsequently becoming indistinguishable from FEN activity at 220 nM. This suggests that an optimal concentration (10 nM) of the enzyme is needed for specific FEN activity.

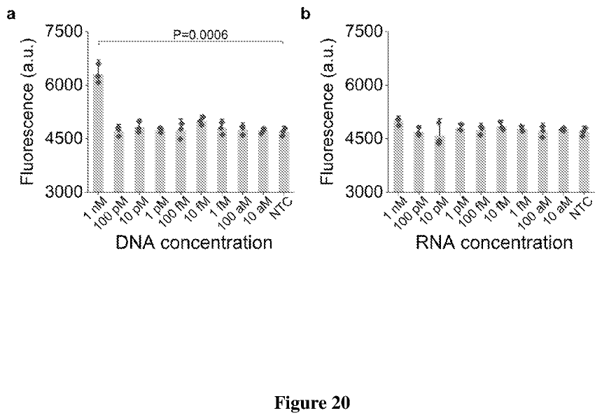

Although FEN can amplify signals through the cyclic annealing and denaturing of the flap primer, it may not be robust enough for the highly sensitive detection of the target in a short time due to the linear amplification of the signal (one flap product formed every cycle per target). To investigate this, the detection limit for both DNA and RNA was ascertained by using 10 nM Tth pol v2. Within 30 min of incubation, up to 100 pM of both DNA and RNA was detected, although the fluorescence signal for 100 pM was slightly higher than that for the no target control (NTC) (FIGS. 13d and e). Increasing the Tth pol v2 concentration to 50 nM reduced the detection limit to 1 nM due to higher non-specific activity resulting in greater background fluorescence (FIG. 20).

### Example 2: Development and Optimization of FELICX

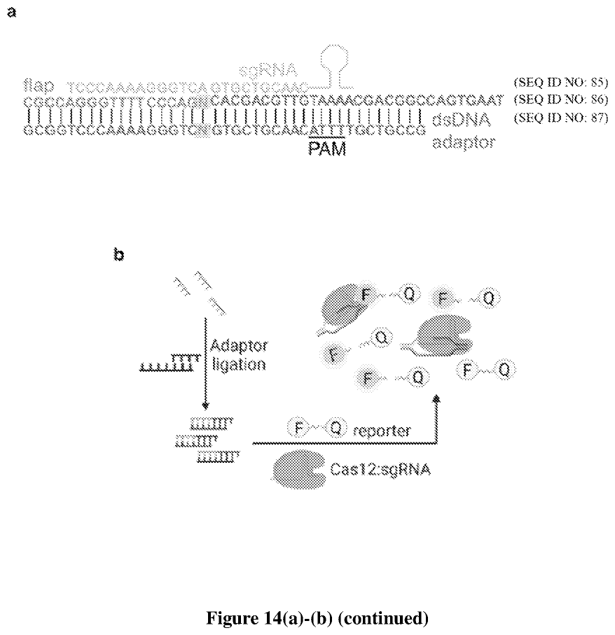

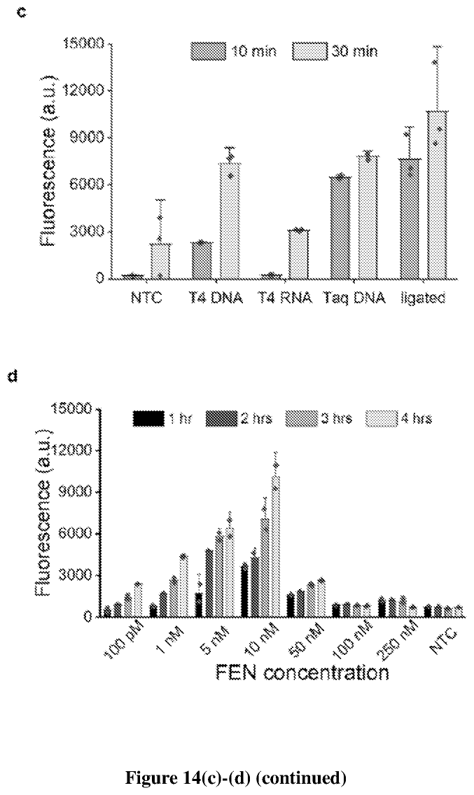

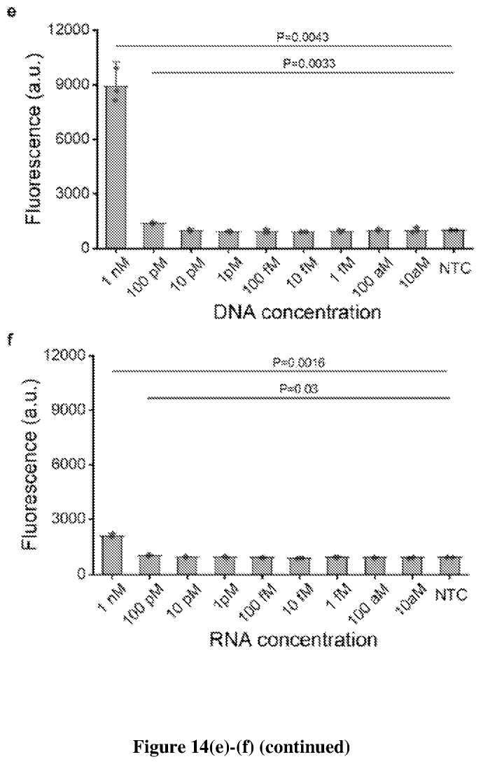

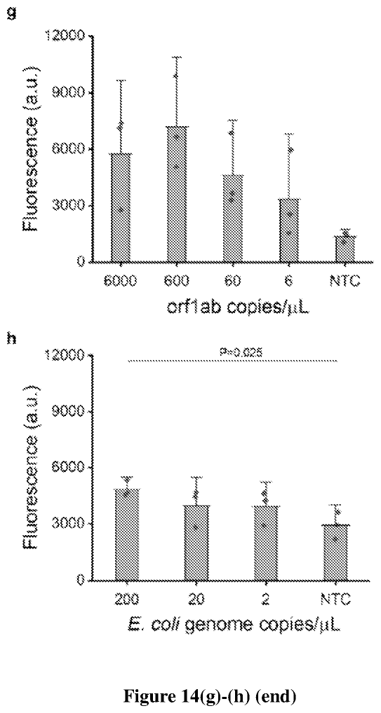

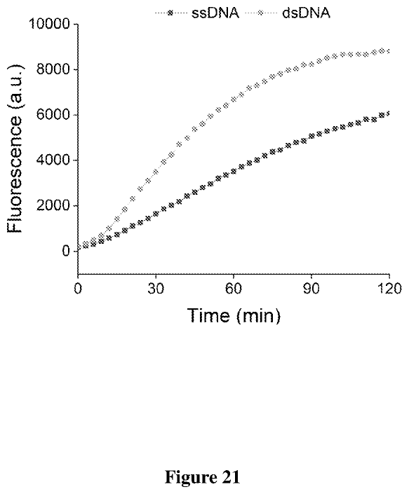

CRISPR-Cas12 acts as an excellent signal amplifier through its rapid trans cleavage activity on ssDNA upon recognition of the DNA substrate. Thus, FEN was combined with CRISPR-Cas12 by converting the flap oligo generated by FEN to the substrate for Cas12 through ligation to an adaptor. For FELICX, Cas12 from Lachnospiraceae bacterium ND2006 was used. As Cas12 has a preference for a dsDNA substrate over ssDNA (FIG. 21), we created a dsDNA adaptor with a 3′ overhang by annealing two oligos: 5′ p-adaptor and ds-convertor. The 5′ p-adaptor has a 5′-phosphate that is ligated to the 3′-OH of the flap oligo by a ligase enzyme, while the ds-convertor carries the PAM adjacent to the binding site of sgRNA (FIG. 14a). The complementary target of the sgRNA is split between the flap and the adaptor, which ensures that neither the adaptor alone nor the uncleaved flap primer produces a signal.

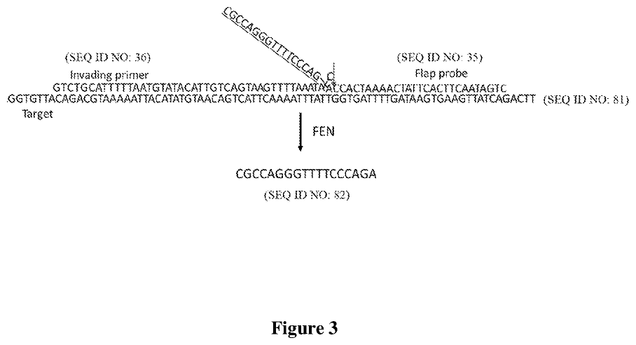

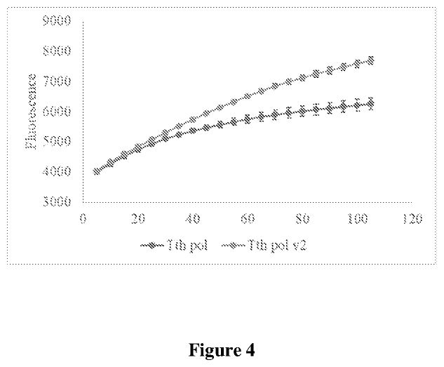

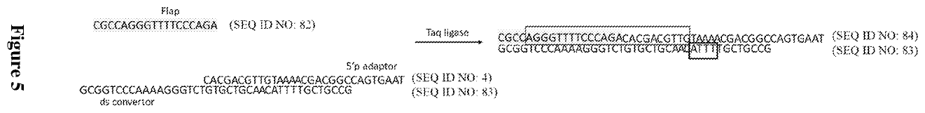

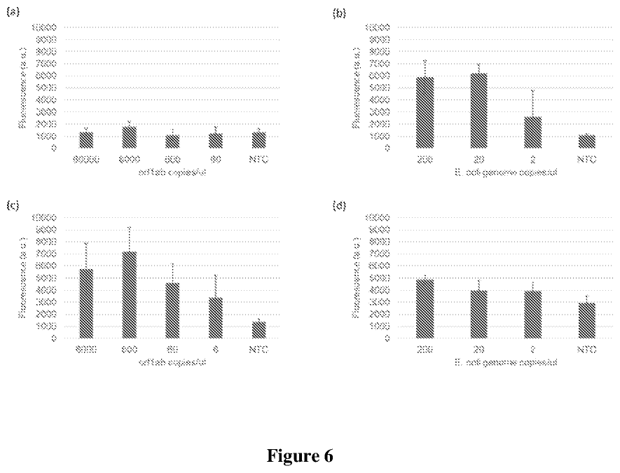

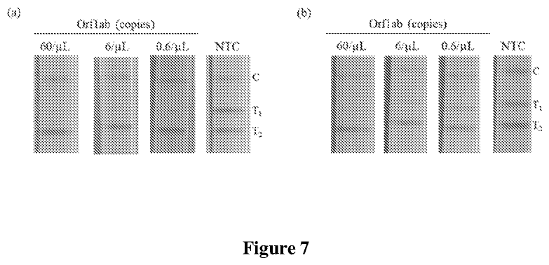

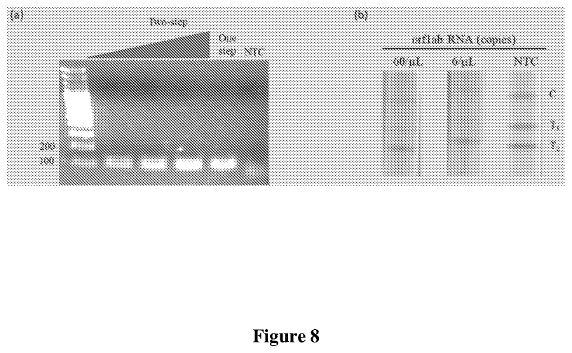

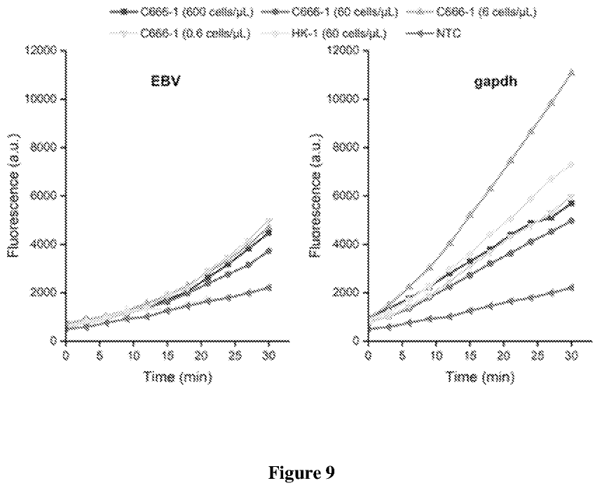

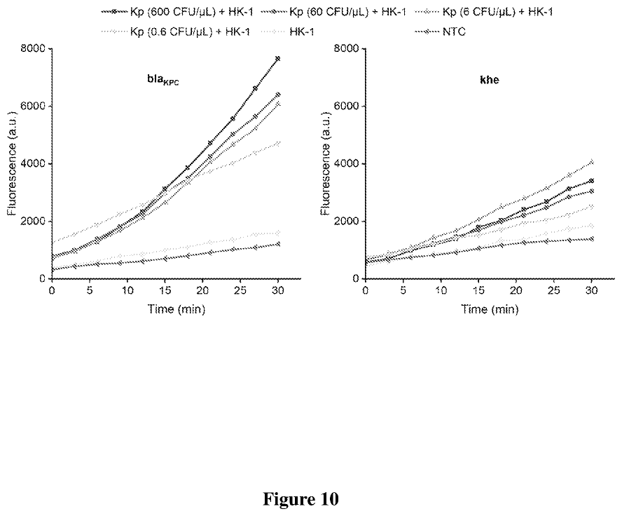

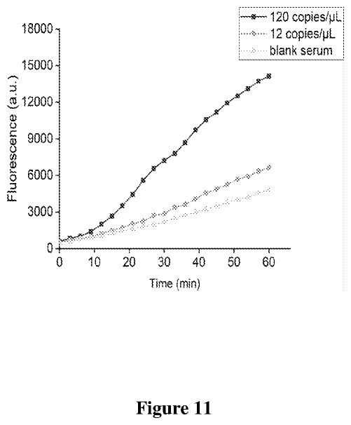

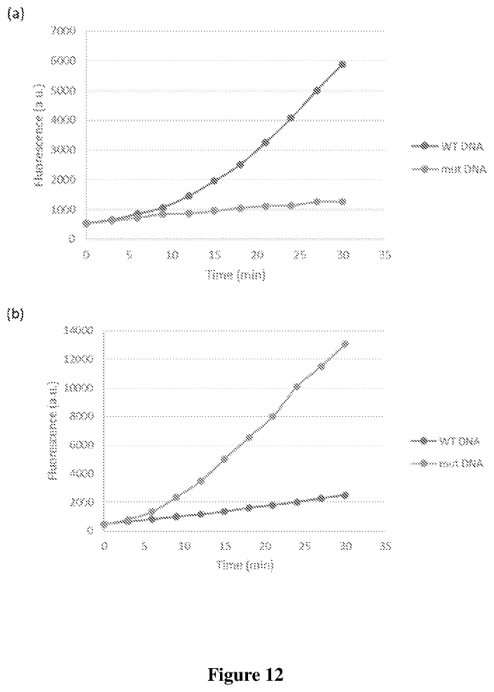

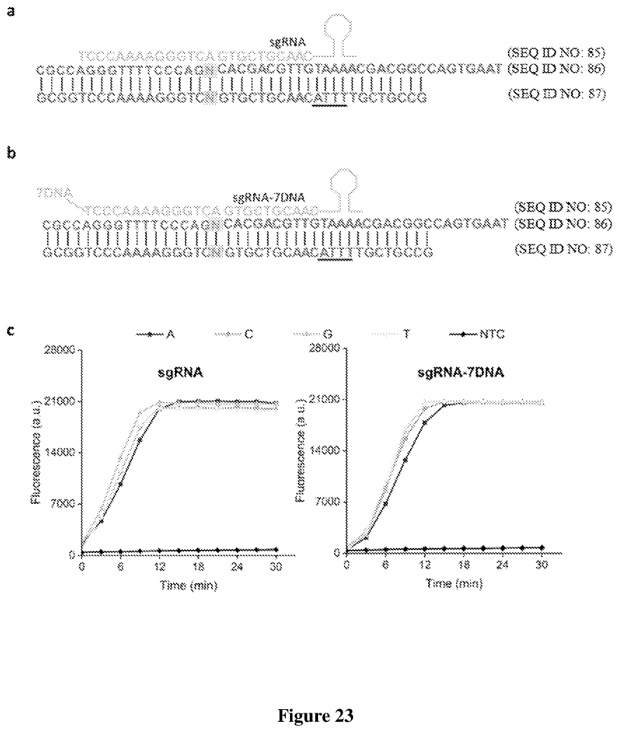

For every new target in FELICX, only the invading primer and the part of the flap primer complementary to the target must be changed (FIG. 13a). The 5′ flap of the flap primer remains constant, as FEN can cleave the flap primer in a sequence-independent manner. However, the cleavage site for FEN is one base pair into the complementary part of the flap primer with the target. Thus, every target can produce a flap with either of the four nucleotides at the 3′ end of the flap, depending on the target sequence. To account for this, the complementary nucleotide in the ds convertor must be changed (see nucleotides N and N′ in FIG. 3a). As this base pair falls within the region recognized by the sgRNA, recognition of the dsDNA adaptor by the Cas12-sgRNA complex was tested with either of the four nucleotides at position N without changing the sgRNA sequence. This would simplify the use of Cas12-sgRNA, eliminating the need for changes in the sgRNA sequence for every new target. For this, Cas12-sgRNA complex with sgRNA complementary to the dsDNA adaptor with thymine at position N was used (FIG. 23a). As shown in FIG. 23c, Cas12-sgRNA detected all possible targets with similar efficiency, irrespective of the nucleotide at the end of the flap. This is expected, as previous reports have shown that LbCas12a can tolerate mismatches in nucleotides 8-18 from the PAM which is the region where the fickle base pair (N-N′) lies. Therefore, for any nucleic acid target, the remaining part of the dsDNA adaptor apart from one base pair and the sgRNA remain unchanged, irrespective of the target sequence, enabling easier reprogramming of FELICX to detect different targets.

For the ligation of the flap to the adaptor, Taq ligase was chosen as its optimal temperature (65° C.) is the same as that of FEN. This would enable a one-pot reaction with both the flap primer cleavage by FEN and adaptor ligation occurring simultaneously. The ligation efficiency of Taq ligase at 65° C. was measured and compared it to that of other ligases, with the reaction performed at the reported optimal temperature of the enzyme: T4 DNA ligase at 21° C. and T4 RNA ligase at 37° C. A synthetic flap oligo was used with the dsDNA adaptor as the substrates and ligation was performed ligation for 30 min. The ligated product was detected by the Cas12-sgRNA complex, with fluorescence signals measured after 10 min or 30 min of incubation at 37° C. (FIG. 14b). Among the three ligases tested, Taq DNA ligase exhibited the highest fluorescence signal within 10 min of incubation with Cas12-sgRNA, which was comparable to the signal of the pre-ligated product (FIG. 14c). Thus, Taq ligase was used as the ligase for FELICX.

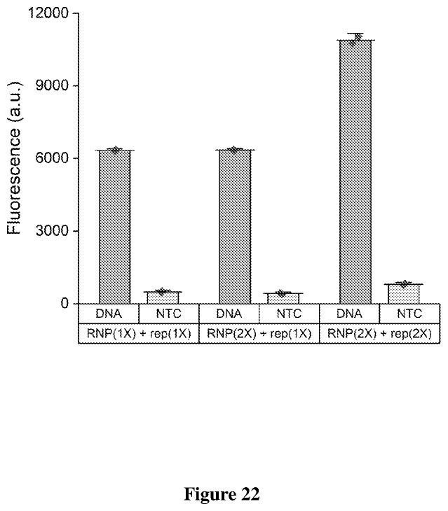

Next, the one-pot FEN+Taq ligase reaction step was optimized by varying the concentration of FEN and the reaction incubation time at 65° C. FIG. 14d shows that the optimal FEN concentration was 10 nM, as it produced the strongest fluorescence signal. Meanwhile, lower and higher FEN concentrations yielded weaker signals. At low concentrations of FEN, fewer flap products are formed, resulting in weaker fluorescence signals. However, at high FEN concentrations, the nonspecific activity of FEN may yield flap products of shorter length that do not anneal to the dsDNA adaptor due to the high reaction temperature or are not recognized by the Cas12-sgRNA complex after ligation due to incomplete hybridization of the sgRNA to the ligated product. Additionally, the fluorescence signal improved with increased incubation time, with 4 h of incubation yielding the strongest signal. To further strengthen the fluorescence signal, the concentrations of both the Cas12-sgRNA complex and the DNA reporter were increased, which led to a significant increase in the specific signal (FIG. 22). A modified sgRNA, extended at the 3′ end with ssDNA, which has previously been reported to increase the trans-cleavage activity of LbCas12a, was also evaluated. However, no improvement in activity was observed compared to that of the unmodified sgRNA (FIG. 23). Using these optimized conditions, the detection limit of DNA and RNA by FELICX was evaluated to determine whether signal amplification by FEN and Cas12 was sufficient for the sensitive detection of nucleic acids. For this, partial orflab DNA or RNA substrates of SARS-CoV-2 were spiked into purified total DNA or RNA from HEK293T cells, respectively, to simulate clinical samples. Despite a prolonged incubation of 4 h at 65° C., only up to 100 PM target was detected by FELICX when both substrates were tested (FIGS. 14e and f). Although this detection limit is similar to that observed with FEN alone (FIGS. 13d and e), the fluorescence signals observed with FELICX were significantly higher compared to FEN alone, indicating signal amplification by Cas12.

Previously reported nucleic acid detection methods have incorporated isothermal nucleic acid amplification techniques, such as loop-mediated isothermal amplification (LAMP) and recombinase polymerase amplification (RPA), to improve detection sensitivity. Target nucleic acids were amplified using LAMP, a commonly used nucleic acid amplification method performed at 65° C., followed by FELICX to detect the partial orflab DNA of SARS-CoV-2 spiked into purified total DNA from HEK293T cells. However, orflab DNA was not detected despite extensive assay optimization.

As non-specific amplification was observed using LAMP with six primers, alternate amplification methods requiring fewer primers, such as RPA and helicase-dependent amplification (HDA), might eliminate the formation of nonspecific products, improving the sensitivity of target detection. HDA was chosen because its working temperature (65° C.) is the same as the temperature of the FEN+Taq ligation step, simplifying the operation of HDA+FELICX. When FELICX was coupled with HDA, six copies/μL (10 aM) of orflab and two copies/μL (3.5 aM) of the E. coli genome were detected in two out of three biological samples (FIGS. 14g and h), a significant improvement compared to LAMP, particularly for orflab detection. Thus, the presence of fewer primers in the nucleic acid amplification step can prevent non-specific product formation and increase target yield, greatly improving its subsequent detection.

Integration of a Lateral Flow Detection System into FELICX

FELICX was integrated with a lateral flow system for signal detection to eliminate the need for a fluorescence reader. PCRD Flex lateral flow strips were used that were capable of detecting two different oligos with conjugated biotin and either FAM or digoxigenin (FIG. 15a). In the final step of FELICX, the Cas12-sgRNA complex cleaves the DNA reporter in the presence of its substrate. To make use of this cleavage activity for the lateral flow strips, the DNA fluorescence reporter was replaced with probes for PCRD Flex. In the presence of the substrate, the Cas12-sgRNA complex cleaves the probe, resulting in the disappearance of the corresponding band on the lateral flow strip (FIG. 15b). Thus, a positive signal for the target nucleic acid is represented by the absence of the band on the lateral flow strip. The partial orflab DNA was used as the target to verify the functionality of the lateral flow strips with FELICX. As the sensitivity of the lateral flow strips is poorer than that of the fluorescence reader, the HDA reaction volume was scaled up from 10 μL to 25 μL. Since high variability and weaker signals were observed when unpurified HDA product was used for FELICX (FIGS. 14g and h), a nucleic acid purification step was included after HDA. The ChargeSwitch PCR Clean-Up Kit was used for purification. When coupled with HDA and subsequent purification, up to 0.6 copies/μL (1 aM) orflab DNA spiked into purified HEK293T DNA was detected using FELICX, as indicated by the disappearing T1 band in the lateral flow strip (FIG. 15c). The total time for detection was 2.5 h, including 1 h of HDA, 1 h of FEN+Taq ligation, and 30 min of the Cas12-sgRNA reaction. There was no loss of sensitivity when the time for HDA+FELICX was reduced to 60 min (20 min each of HDA, FEN+Taq ligation, and Cas12-sgRNA) (FIG. 15d), demonstrating the robustness of HDA+FELICX.

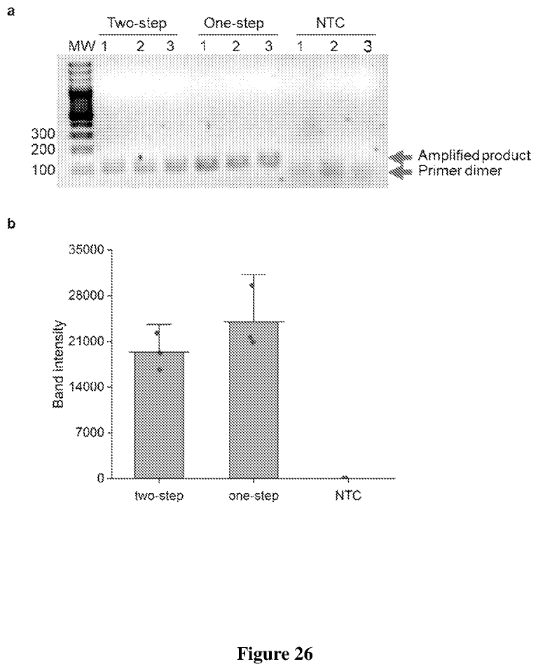

Having confirmed the applicability of this method for rapid and sensitive detection of DNA. HDA+FELICX was used to detect RNA targets. As the HDA reaction mixture lacks a dedicated reverse transcriptase, the WarmStart RTx reverse transcriptase was used due to its optimal temperature of 65° C. and similar buffer requirements as HDA. As RTx has not been used previously with HDA, we evaluated the functioning of RTx+HDA in a one-step (one-pot RTx+HDA) or two-step (sequential RTx and HDA) reaction to determine whether RTx interferes with the HDA reaction. There was no difference observed between the reactions (FIG. 26), and the one-step RTx+HAD was chosen due to its lower complexity. RTx+HDA was performed, followed by FELICX to detect orflab RNA spiked into purified HEK293T RNA. 0.6 copies/μL RNA was detected in the process (FIG. 15e). It took ˜90 min in total to detect the RNA (45 min for RTx+HDA, 20 min for FEN+Taq ligation, and 20 min for Cas12-sgRNA). These results demonstrate that HDA+FELICX can detect both DNA and RNA using lateral flow strips with similar sensitivities in 60 min and 90 min, respectively.

### Example 3: Detection of Polynucleotide Analytes Using FELICX

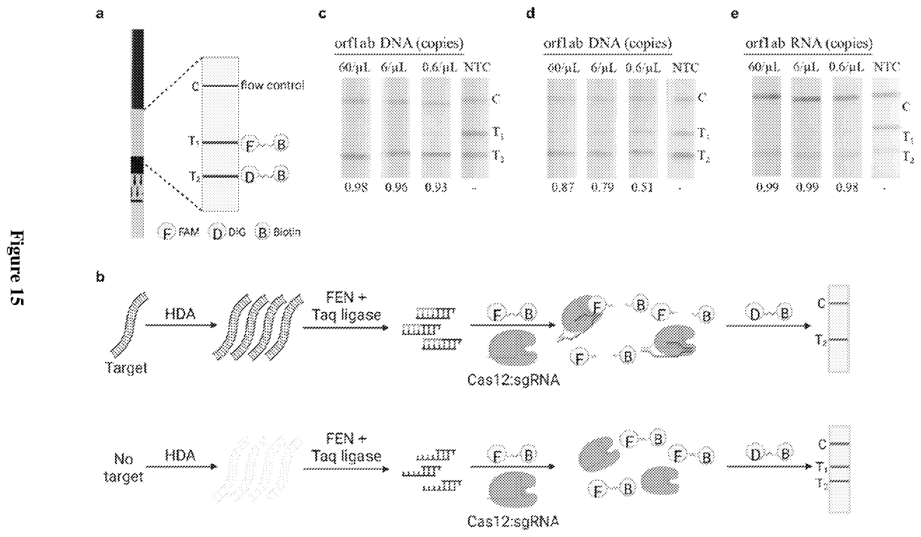

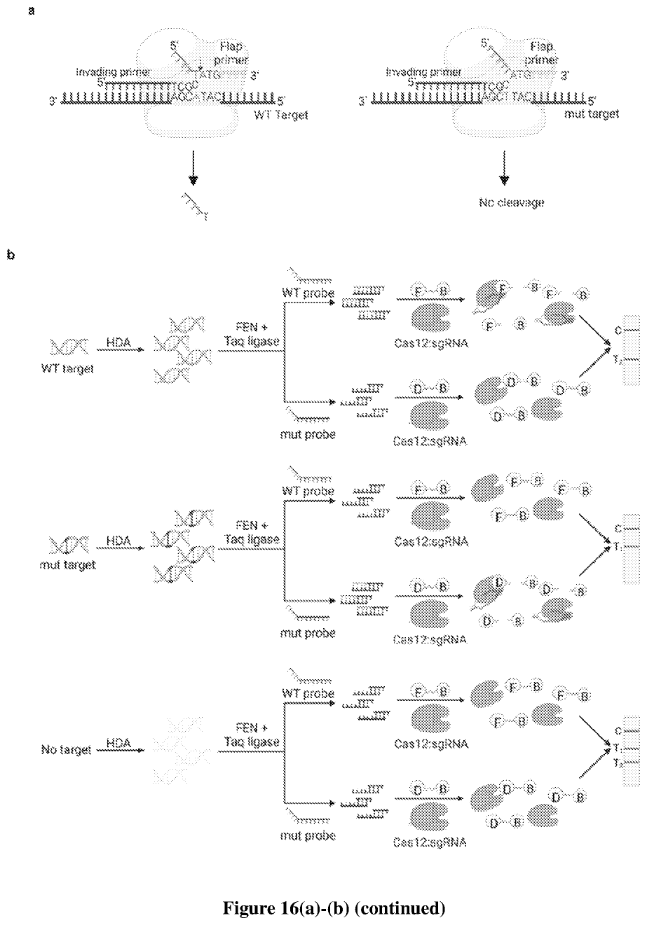

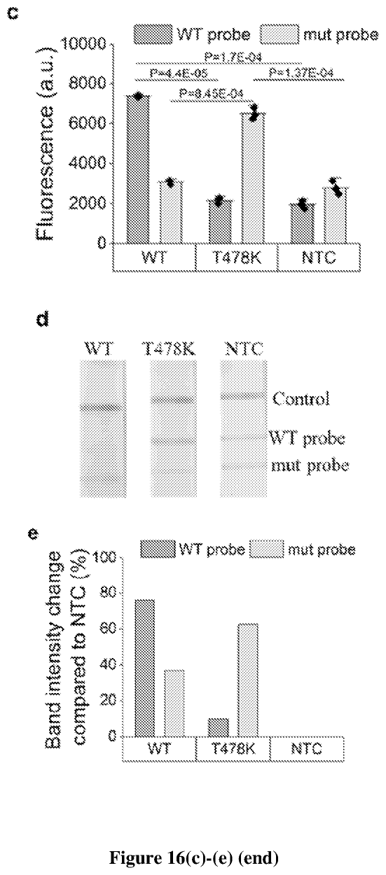

Detecting SNPs is important for disease diagnosis and treatment, as well as for identifying pathogen variants. FEN can be used to distinguish between SNPs by designing the flap and invading primers such that the one base-pair overlap between the primers falls at the SNP position (FIG. 16a). If there is an SNP in the target, the absence of overlap between the flap and the invading primers will abolish the activity of FEN. Thus, the absence or presence of the SNP in the target sample can be confirmed by probing the sample with flap primers specific to the wild type and the variant target sequence (FIG. 16b). To this end, FELICX was used to distinguish between the receptor-binding domain in the spike protein of wild-type SARS CoV-2 and its variant carrying the T478K mutation, which is found in the Delta and Omicron variants of the virus. Flap primers, namely, the WT and mut probes, specific to the wild-type (WT) sequence and the variant, respectively, were designed and used to probe both targets. In the case of the WT target, after FELICX, there was an ˜2.5-fold higher fluorescence signal with the WT probe compared to the mut probe (FIG. 16c). In contrast, the variant target with the T478K mutation showed an ˜2.5-fold higher fluorescence signal with the mut probe than with the WT probe. Although the difference between the WT and T478K variants was only a single base pair (with the ACG codon changed to AAG), FELICX easily detected the SNP, as indicated by the opposite trend in the fluorescence signal observed for the WT and the variant sequence.

Detection of SNPs is also demonstrated with the PCRD Flex strips, with each test line corresponding to either the WT or mut probes. For this, a FAM-biotin reporter was used for the WT probe and a DIG-biotin reporter for the mut probe (FIG. 16b). Similar to the result shown in FIG. 16c, the WT sample showed a positive signal with the WT probe, as indicated by the disappearance of the band corresponding to this probe (FIGS. 16d and e). For the T478K variant, the band corresponding to the mut probe disappeared, indicating that a positive signal was obtained with this probe.

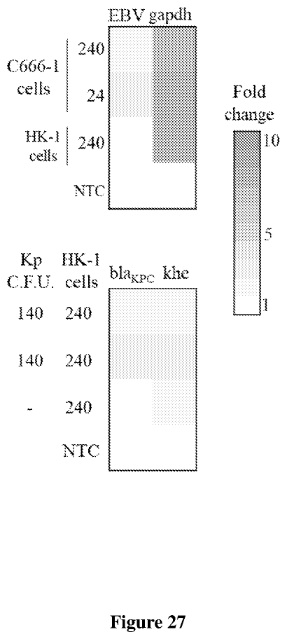

FELICX can also detect other pathogens of interest in more complex samples, e.g., EBV, which causes infectious mononucleosis and is associated with autoimmune diseases, nasopharyngeal carcinoma (NPC), and other neoplasms. C666-1, an NPC cell line and a natural host of EBV, was used. The EBV-negative HK-1 NPC cell line was also included in the analysis, with the housekeeping gene gapdh used as the internal control for assay functionality. Cell lysis followed by RTx+HDA and FELICX were performe, and both EBV and gapdh were detected at levels as low as 0.6 cells/μL of C666-1 (FIGS. 17a and 27, top panel), demonstrating highly sensitive EBV detection by HDA+FELICX. The sensitive detection of the target in whole cells using HDA+FELICX validated the robustness of the presentmethod.

FELICX could also detect K. pneumoniae, the most common nosocomial pathogen and a major source of patient complications worldwide. K. pneumoniae is known to be a causative agent for neonatal sepsis, pneumonia, surgical wound infection, and cystitis. In addition to its wide prevalence, K. pneumoniae is also a major source of antibiotic resistance genes, particularly those encoding carbapenemases. Detecting the pathogen in clinical samples and definitively identifying its antibiotic resistance status is therefore essential for guiding infection treatment. HDA+FELICX was used to detect the carbapenemase resistance gene (blaKPC) in pathogenic K. pneumoniae DSM 2026 bacterial cells spiked into whole HK-1 cells. The hemolysin gene (khe), which is present in all K. pneumoniae isolates, was used as an internal control. Using similar conditions as those described for EBV detection, bla_{KPC} and khe were detected in up to 0.6 c.f. u./μL K. pneumoniae DSM2026 (FIGS. 17b and 27, bottom panel). No signal was observed for either gene in HK-1 cells alone. As RTx was not used in this assay, only the copies of the genes in the K. pneumonia DSM2026 genome were detected, underscoring the assay's high detection sensitivity.

Tth-pol v2 sequence
(SEQ ID NO: 1)
MEAMLPLFEPKGRVLLVDGHHLAYRTFFALKGLTTSRGEPVQAVY
GFAKSLLKALKEDGYKAVFVVFDAKAPSFRHEAYEAYKAGRAPTP
EDFPRQLALIKELVDLLGFTRLEVPGYEADDVLATLAKKAEKEGY
EVRILTADRDLYQLVSDRVAVLHPEGHLITPEWLWEKYGLRPEQW
VDFRALVGDPSDNLPGVKGIGEKTALKLLKEWGSLENLLKNLDRV
KPENVREKIKAHLEDLRLSLELSRVRTDLPLEVDLAQGREPDREG
LRAFLERLEFGSLLHEFGLLEAPAPLEEAPWPPPEGAFVGFVLSR
PEPMWAELKALAACRDGRVHRAADPLAGLKDLKEVRGLLAKDLAV
LASREGLDLVPGDDPMLLAYLLDPSNTTPEGVARRYGGEWTEDAA
HRALLSERLHRNLLKRLEGEEKLLWLYHEVEKPLSRVLAHMEATG
VRLDVAYLQALSLELAEEIRRLEEEVFRLAGHPFNLNSRDQLERV
LFDELRLPALKKTKKTGKRSTSAAVLEALREAHPIVEKILQHREL
TKLKNTYVDPLPSLVHPRTGRLHTRFNQTATATGRLSSSDPNLQN
IPVRTPLGQRIRRAFVAEVGWALVVLDYSQMGLRVLAHLSGDENL
IRVFQEGKDIHTQTASWMFGVPPEAVDPLMRRAAKTVNFGVLYGM
SAHRLSQELAIPYEEAVAFIERYFQSFPKVRAWIEKTLEEGRKRG
YVETLFGRRRYVPDLNARVKSVREAAERMAFNMPVQGTAADLMKL
AMVKLFPRLREMGARMLLQVADELLLEAPQARAEEVAALAKEAME
KAYPLAVPLEVEVGMGEDWLSAKGVSGWRLFKKISHHHHHH
Tth-pol v3 sequence
(SEQ ID NO: 2)
MEAMLPLFEPKGRVLLVDGHHLAYRTFFALKGLTTSRGEPVQAVY
GFAKSLLKALKEDGYKAVFVVFDAKAPSFRHEAYEAYKAGRAPTP
EDFPRQLALIKELVDLLGFTRLEVPGYEADDVLATLAKKAEKEGY
EVRILTADRDLYQLVSDRVAVLHPEGHLITPEWLWEKYGLRPEQW
VDFRALVGDPSDNLPGVKGIGEKTALKLLKEWGSLENLLKNLDRV
KPENVREKIKAHLEDLRLSLELSRVRTDLPLEVDLAQGREPDREG
LRAFLERLEFGSLLHEFGLLEAPAPLEEAPWPPPEGAFVGFVLSR
PEPMWAELKALAACRDGRVHRAADPLAGLKDLKEVRGLLAKDLAV
LASREGLDLVPGDDPMLLAYLLDPSNTTPEGVARRYGGEWTEDAA
HRALLSERLHRNLLKRLEGEEKLLWLYHEVEKPLSRVLAHMEATG
VRLDVAYLQALSLELAEEIRRLEEEVFRLAGHPFNLNSRDQLERV
LFDELRLPALKKTKKTGKRSTSAAVLEALREAHPIVEKILQHREL
TKLKNTYVDPLPSLVHPRTGRLHTRFNQTATATGRLSSSDPNLQN
IPVRTPLGQRIRRTFVAEAGRQLVALDYSQTELRVLAHLSGDENL
IRVFQEGKDIHTQTASWMFGVPPEAVDPLMRRAAKTVNFGVLYGM
SAHRLSQELAIPYEEAVAFIERYFQSFPKVRAWIEKTLEEGRKRG
YVETLFGRRRYVPDLNARVKSVREAAERMAFNMPVQGTAADLMKL
AMVKLFPRLREMGARMLLQVADELLLEAPQARAEEVAALAKEAME
KAYPLAVPLEVEVGMGEDWLSAKGVSGWRLFKKISHHHHHH
Cas12 gRNA
(SEQ ID NO: 3)
5′-TAATTTCTACTAAGTGTAGATCAACGTCGTGACTGGGAAAACCCT-3′

TABLE 1
Oligos used in this study

| Name | Sequence | | Remarks |
|---|---|---|---|
| 5′p adaptor | P- | | Annealed to form |
| | CACGACGTTGTAAAACGACGGCCAGTGAA | | dsDNA adaptor |
| | T (SEQ ID NO: 4) | | |
| ds- | GCCGTCGTTTTACAACGTCGTGNCTGGGA | | |
| convertor | AAACCCTGGCG (SEQ ID NO: 5) | | |
| lig pos N | CGCCAGGGTTTTCCCAGNCACGACGTTGT | | Control oligos |
| | AAAACGACGGCCAGTG (SEQ ID NO: 6) | | representing ligated |
| | | | flap oligo with 5′p |
| | | | adaptor. Used to create |
| | | | pre-ligated dsDNA |
| | | | adaptor |
| Flap oligo | CGCCAGGGTTTTCCCAGT (SEQ ID NO: 7) | | Used as a positive |
| | | | control for the FEN |
| | | | reaction |
| fluorophore | 6-FAM-X1-IABKFQ | X1: TTATTATTAT | Cas12-sgRNA |
| -quencher | | (SEQ ID NO: 8) | reporters |
| reporter | | | |
| FAM-Bio | 6-FAM-X1-BIO | | |
| reporter | | | |
| DIG-Bio | DIG-X1-BIO | | |
| reporter | | | |
| Spike- | AAATTTACCAAGCAGGCAGTAAGCCATGT | | For mutagenesis of the |
| T478K-F | AACGGAGTAGAGGG (SEQ ID NO: 9) | | RBD of spike protein |
| Spike- | TTACTGCCTGCTTGGTAAATTTCGGTTGAA | | of SARS-CoV-2 |
| T478K-R | ATATCCCGCTCAAAAGG (SEQ ID NO: 10) | | |
| Spike-F | TAATACGACTCACTATAGGGAGA (SEQ ID | | Amplification of RBD |
| | NO: 11) | | of spike protein of |
| Spike-R | GATCAGCGGGTTTAAACG (SEQ ID NO: 12) | | SARS-CoV-2 |
| Scgb1-F | ATGAAGTTGCTGATGGTCCTCATGCTGGCG | | Overlapping oligos to |
| | GCCCTCTCCCAGCACTGCTACGCAGGCTCT | | form the template for |
| | GGCTGCCCCTTATTGGAGAATGTGATTTCC | | SCGB2A2 |
| | (SEQ ID NO: 13) | | |
| Scgb2-R | TGTCGTCTATGAACTCTTGAAGAAGTTCTT | | |
| | TGTATTCAGTCTTAGACACTTGTGGATTGA | | |
| | TTGTCTTGGAAATCACATTCTCCAATAAGG | | |
| | (SEQ ID NO: 14) | | |
| Scgb3-F | TCAAGAGTTCATAGACGACAATGCCACTA | | |
| | CAAATGCCATAGATGAATTGAAGGAATGT | | |
| | TTTCTTAACCAAACGGATGAAACTCTGAG | | |
| | CAA (SEQ ID NO: 15) | | |
| Scgb4-R | TTAAAATAAATCACAAAGACTGCTGTCAT | | |
| | ATATTAATTGCATAAACACCTCAACATTGC | | |
| | TCAGAGTTTCATCCG (SEQ ID NO: 16) | | |
| Scgb2a2- | TAATACGACTCACTATAGGGATGAAGTTG | | Oligos to amplify and |
| T7-F | CTGATGGTC (SEQ ID NO: 17) | | form the template for |
| Scgb2a2-R | TTAAAATAAATCACAAAGACTGCTGTC | | IVT of SCGB2A2 |
| | (SEQ ID NO: 18) | | |
| Flap primer | 6- | X2: CCCAGTTGT | Primers to evaluate |
| with | FAM/X2/ZEN/X3/ | X3: CTGATTGTC | FEN activity on |
| fluorophore | 3IABKFQ/ | CTCACT | orflab. Part of the |
| and quencher | | (SEQ ID NO: 19) | primer not |

| | | |
|---|---|---|
| Invading | ATCTCTAATTGAGGTTGAACCTCAACAATT | complementary to |
| primer | GTTTGAATAGTAGTA (SEQ ID NO: 20) | orflab is underlined. |
| Invading | CATCTCTAATTGAGGTTGAACCTCAACAAT | |
| primer with | TGTTTGAATAGTAGACCGTCACGCCAGTC | |
| 3′ flap | (SEQ ID NO: 21) | |

TABLE 2
LAMP primers used in this study

| Primer | Sequence | |
|---|---|---|
| Set 1 | FIP | GTTGTCTGATTGTCCTCACTGCGAAGAGCAAGAAGAAG |
| for | | ATTGGT (SEQ ID NO: 22) |
| orflab | BIP | ATGCAGACATTGTGGAAGAAGCTGCAACACCTCCTCCA |
| | | TGTT (SEQ ID NO: 23) |
| | F3 | GCCACTTCTGCTGCTCTTCAA (SEQ ID NO: 24) |
| | B3 | CTTGCATGGCATTGTTAGTAGC (SEQ ID NO: 25) |
| | LF | CGTCTTGTTGACCAACAGTTTG (SEQ ID NO: 26) |
| | LB | ACAGTGGTTGTTAATGCAGCCA (SEQ ID NO: 27) |
| | Swarm F1 | GTTGTCTGATTGTCCTCACTGC (SEQ ID NO: 28) |
| | Swarm B1 | TGCAGACATTGTGGAAGAAGC (SEQ ID NO: 29) |
| Set 2 | FIP | ACCAACAGTTTGTTGACTATCATCACTGCTGCTCTT |
| for | | CAACCTG (SEQ ID NO: 30) |
| orflab | BIP | CGGCAGTGAGGACAATCAGACACTGGTGTAAGTTCCAT |
| | | CTC (SEQ ID NO: 31) |
| | F3 | TGGAATTTGGTGCCACTT (SEQ ID NO: 32) |
| | B3 | CTATTCACTTCAATAGTCTGAACA (SEQ ID NO: 33) |
| | LF | CAATCTTCTTCTTGCTCTTCTT (SEQ ID NO: 34) |
| | LB | TTCAAACAATTGTTGAGGTTCAACC (SEQ ID NO: 35) |
| Set 3 | FIP | CAATAGTCTGAACAACTGGTGTAAGTCAAACAATTGTTG |
| for | | AGGTTC (SEQ ID NO: 36) |
| orflab | BIP | TGCAGACATTGTGGAAGAAGCATTGGCTGCATTAACAA |
| | | CC (SEQ ID NO: 37) |
| | F3 | TGAGGACAATCAGACAACT (SEQ ID NO: 38) |
| | B3 | CTCCTCCATGTTTAAGGTAA (SEQ ID NO: 39) |
| | LF | CCATCTCTAATTGAGGTT (SEQ ID NO: 40) |
| | LB | AAGGTAAAACCAACAGT (SEQ ID NO: 41) |
| Set 4 | FIP | CCTTGGTAATCATCTTCAGTACCATGAAGAAGAAGGTG |
| for | | ATTGTGAA (SEQ ID NO: 42) |
| orflab | BIP | TTGGAATTTGGTGCCACTTCTGTTTGTTGACTATCATCAT |
| | | CTAACC (SEQ ID NO: 43) |
| | F3 | CTACCCTCCAGATGAGGAT (SEQ ID NO: 44) |
| | B3 | CACTGCCGTCTTGTTGAC (SEQ ID NO: 45) |
| | LF | CTCATATTGAGTTGATGGCTCAAAC (SEQ ID NO: 46) |
| | LB | CAACCTGAAGAAGAGCAAGAAGA (SEQ ID NO: 47) |

TABLE 3
HDA primers used in this study

| Template | Primer sequence | |
|---|---|---|
| orflab | F | GGAACTTACACCAGTTGTTCAGACTATT (SEQ ID NO: 48) |
| | R | TAGCTTCTTCCACAATGTCTGCATTT (SEQ ID NO: 49) |
| malB in | F | GCTGTCGATGACAGGTTGTTACAAAG (SEQ ID NO: 50) |
| E. coli | R | GAGATTGATATCTTTCGATACCACGACCT (SEQ ID NO: 51) |
| RBD in | F | TTTGAGCGGGATATTTCAACCGAA (SEQ ID NO: 52) |
| the spike | R | GCCATAAGATTGAAGAGGAAAGTAGCA (SEQ ID NO: 53) |
| protein | | |
| EBV | F | CCTCTATTCCTTTGCTCTCATGCTTATT (SEQ ID NO: 54) |
| | R | GAGTAGACCAAGGCCTCCAAGT (SEQ ID NO: 55) |
| gapdh | F | CCTTCATTGACCTCAACTACATGGTTTAC (SEQ ID NO: 56) |
| | R | CATTGATGACAAGCTTCCCGTTCTC (SEQ ID NO: 57) |
| bla_{KPC} | F | CCCGCAGCCGCTTGAGCAAATTA (SEQ ID NO: 58) |
| | R | GCCGCTGGCCAGATCCATTTCTATC (SEQ ID NO: 59) |
| khe | F | GCCGGGATTGAGCGGGTAATAAAT (SEQ ID NO: 60) |
| | R | CCTTTCTCAATAACACCGAGCAGGAAG (SEQ ID NO: 61) |
| SCGB2A2 | F | CAATGCCACTACAAATGCCATAGATGAA (SEQ ID NO: 62) |
| | R | GACTGCTGTCATATATTAATTGCATAAACACCTC (SEQ ID |
| | | NO: 63) |

Table 4: Flap and invading primers used in this study. The constant part of the flap primer is underlined. Boxed is the part of the invading primer that is not complementary.

| Target | Flap primer sequence | Invading primer sequence |
|---|---|---|
| orflab | CGCCAGGGTTTTCCCAGACCAC | GTCTGCATTTTTAATGTATACAT |
| | TAAAACTATTCACTTCAATAGT | 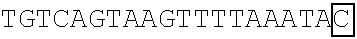 |
| | C (SEQ ID NO: 64) | (SEQ ID NO: 65) |
| malB | CGCCAGGGTTTTCCCAGGCCAT | CCAGGCTTTCGTTACATTTTGCA |
| | GCCCTTCTCCC (SEQ ID NO: 66) | 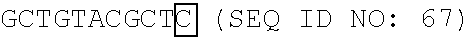 |
| RBD in | WT:CGCCAGGGTTTTCCCAGGT | TAAATCCCTCTACTCCGTTACAT |
| the spike | ACTGCCTGCTTG (SEQ ID NO: | 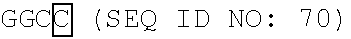 |
| protein | 68) | |
| | Mut: | |
| | CGCCAGGGTTTTCCCAGTTACT | |
| | GCCTGCTTG (SEQ ID NO: 69) | |
| EBV | CGCCAGGGTTTTCCCAGCTCTT | GCCTCCAAGTGGACAGAGAAGG |
| | CTGAAGATAAAGATGATG | 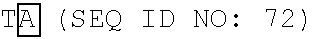 |
| | (SEQ ID NO: 71) | |
| gapdh | CGCCAGGGTTTTCCCAGTTGAC | GACAAGCTTCCCGTTCTCAGCC |
| | GGTGCCAT (SEQ ID NO: 73) | 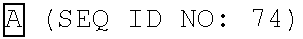 |
| blaKPC | CGCCAGGGTTTTCCCAGTACGC | TGGCCAGATCCATTTCTATCATG |
| | GGCCCGAC (SEQ ID NO: 75) | 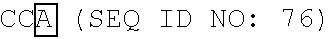 |
| khe | CGCCAGGGTTTTCCCAGTGCTG | 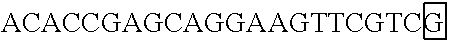 |
| | GTGGTGG (SEQ ID NO: 77) | (SEQ ID NO: 78) |
| SCGB2A2 | CGCCAGGGTTTTCCCAGTCAGA | GACTGCTGTCATATATTAATTGC |
| | GTTTCATCCGTTTGG (SEQ ID | 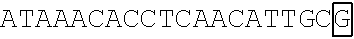 |
| | NO: 79) | (SEQ ID NO: 80) |

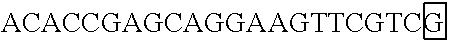

1. A method of detecting a polynucleotide analyte in a sample, the method comprising:
a) contacting the sample comprising the polynucleotide analyte with:
i) a first nucleic acid probe comprising a 3′ portion complementary to a first portion of the polynucleotide analyte and a 5′ portion that is not complementary to and does not hybridize to the polynucleotide analyte;
ii) a second nucleic acid probe comprising a 5′ portion complementary to a second portion of said polynucleotide analyte and a 3′ portion that is not complementary to and does not hybridize to the polynucleotide analyte, wherein said first portion of the polynucleotide analyte is 5′ to and contiguous with the second portion of the polynucleotide analyte; and
iii) a structure-specific nucleic acid cleaving agent;
wherein hybridization of the first nucleic acid probe to the first portion of the polynucleotide analyte and hybridization of the second nucleic acid probe to the second portion of the polynucleotide analyte forms a cleavage structure; and
wherein formation of the cleavage structure allows cleavage of the first nucleic acid probe by the cleaving agent to release a 5′ flap comprising the 5′ portion of the first nucleic acid probe that is not complementary to and does not hybridize to the polynucleotide analyte;
b) ligating the 5′ flap to a nucleic acid adaptor to form an adaptor-ligated product, wherein the nucleic acid adaptor comprises a double-stranded region and a 3′overhang extension that is complementary to the 5′ flap and wherein the 3′ overhang extension hybridizes to the 5′ flap;
c) contacting the adaptor-ligated product with:
i) a type V CRISPR/Cas effector protein;
ii) a guide RNA comprising a region that binds to the type V CRISPR/Cas effector protein and a guide sequence that is complementary to a portion of the 5′ flap and a portion of the nucleic acid adaptor that is ligated to and adjacent to the 5′ flap; and
iii) a single stranded detector DNA;
and
d) measuring a detectable signal produced by cleavage of the single stranded detector DNA by the type V CRISPR/Cas effector protein to detect the adaptor-ligated product, thereby detecting the polynucleotide analyte in the sample.

2-22. (canceled)

23. The method of claim 1, wherein the polynucleotide analyte is a DNA or an RNA.

24. The method of claim 1, wherein the polynucleotide analyte is single stranded or double stranded.

25. The method of claim 1, wherein the method further comprises amplifying the polynucleotide analyte before step (a).

26. The method of claim 1, wherein the polynucleotide analyte comprises a single nucleotide polymorphism (SNP).

27. The method of claim 1, wherein the polynucleotide analyte is a viral nucleic acid.

28. The method of claim 1, wherein the cleaving agent of step a) is an enzyme with flap endonuclease activity.

29. The method of claim 28, wherein the enzyme with flap endonuclease activity is a DNA polymerase from Thermus thermophilus.

30. The method of claim 28, wherein the enzyme with flap endonuclease activity comprises an amino acid sequence having at least 80% sequence identity to an amino acid sequence set forth in SEQ ID NO: 1.

31. The method of claim 1, wherein step c) is performed in the presence of a ligase.

32. The method of claim 31, wherein the ligase is a thermostable ligase.

33. The method of claim 1, wherein the type V CRISPR/Cas effector protein is a Cas12 protein.

34. The method of claim 1, wherein the single stranded detector DNA comprises a fluorophore-quencher pair.

35. The method of claim 1, wherein the cleavage of the single stranded detector DNA is detected visually.

36. A method of detecting a single nucleotide polymorphism (SNP) in a polynucleotide analyte in a sample, the method comprising:
a) contacting the sample comprising the polynucleotide analyte with:
i) a first nucleic acid probe comprising a 3′ portion complementary to a first portion of the polynucleotide analyte and a 5′ portion that is not complementary to and does not hybridize to the polynucleotide analyte;
ii) a second nucleic acid probe comprising a 5′ portion complementary to a second portion of said polynucleotide analyte and a 3′ portion that is not complementary to and does not hybridize to the polynucleotide analyte, wherein said first portion of the polynucleotide analyte is 5′ to and contiguous with the second portion of the polynucleotide analyte; and
iii) a structure-specific nucleic acid cleaving agent;
wherein hybridization of the first nucleic acid probe to the first portion of the polynucleotide analyte and hybridization of the second nucleic acid probe to the second portion of the polynucleotide analyte forms a cleavage structure; and
wherein formation of the cleavage structure allows cleavage of the first nucleic acid probe by the cleaving agent to release a 5′ flap comprising the 5′ portion of the first nucleic acid probe that is not complementary to and does not hybridize to the polynucleotide analyte;
b) ligating the 5′ flap to a nucleic acid adaptor to form an adaptor-ligated product, wherein the nucleic acid adaptor comprises a double-stranded region and a 3′overhang extension that is complementary to the 5′ flap and wherein the 3′ overhang extension hybridizes to the 5′ flap;
c) contacting the adaptor-ligated product with:
i) a type V CRISPR/Cas effector protein;
ii) a guide RNA comprising a region that binds to the type V CRISPR/Cas effector protein and a guide sequence that is complementary to a portion of the 5′ flap and a portion of the nucleic acid adaptor that is ligated to and adjacent to the 5′ flap; and
iii) a single stranded detector DNA;
and
d) measuring a detectable signal produced by cleavage of the single stranded detector DNA by the type V CRISPR/Cas effector protein to detect the adaptor-ligated product, thereby detecting the SNP in the polynucleotide analyte in the sample.

37. A method of detecting a polynucleotide analyte in a sample, the method comprising
a) i) contacting the sample comprising the polynucleotide analyte with a first nucleic acid probe and a second nucleic acid probe that are configured to form a cleavage structure in the presence of the polynucleotide analyte; and
ii) a structure-specific nucleic acid cleaving agent;
wherein formation of the cleavage structure allows cleavage of the first nucleic acid probe by the cleaving agent to release a 5′ flap from the first nucleic acid probe;
b) ligating the 5′ flap to a nucleic acid adaptor to form an adaptor-ligated product, wherein the nucleic acid adaptor comprises a double-stranded region and a 3′overhang extension that is complementary to the 5′ flap and wherein the 3′ overhang extension hybridizes to the 5′ flap to the nucleic acid adaptor;
c) contacting the adaptor-ligated product with:
i) a type V CRISPR/Cas effector protein;
ii) a guide RNA comprising a region that binds to the type V CRISPR/Cas effector protein and a guide sequence that is complementary to a portion of the 5′ flap and a portion of the nucleic acid adaptor that is ligated to and adjacent to the 5′ flap; and
iii) a single stranded detector DNA; and
d) measuring a detectable signal produced by cleavage of the single stranded detector DNA by the type V CRISPR/Cas effector protein to detect the adaptor-ligated product, thereby detecting the polynucleotide analyte in the sample.

38. A kit for detecting a polynucleotide analyte in a sample, comprising a structure-specific nucleic acid cleaving agent, a nucleic acid ligase and a type V CRISPR/Cas effector protein.

39. The kit of claim 38, wherein the structure-specific nucleic acid cleaving agent is a polypeptide comprising an amino acid sequence having at least 80% sequence identity to an amino acid sequence set forth in SEQ ID NO: 1.

40. The kit of claim 38, further comprising a first nucleic acid probe and a second nucleic acid probe, wherein the first and second nucleic acid probes are configured to form a cleavage structure in the presence of the polynucleotide analyte.

41. The kit of claim 38, further comprising a first adaptor polynucleotide and a second adaptor polynucleotide, wherein the first and second adaptor polynucleotides are configured to form a nucleic acid adaptor on hybridization.

42. The kit of claim 41, further comprising a guide RNA configured to bind to the type V CRISPR/Cas effector protein and a product of ligation of the nucleic acid adaptor and a cleavage product of the structure-specific nucleic acid cleaving agent.

43. The kit of claim 38, further comprising a single stranded detector DNA.
